(19) United States
(12) Patent Application Publication
(10) Pub. No.: US 2026/0070114 A1
(43) Pub. Date: Mar. 12, 2026

(54) COMPRESSION COLLAR EXPANDER ASSEMBLY AND METHOD OF USE

(71) Applicant: Life Technologies Corporation, Carlsbad, CA (US)

(72) Inventors: Michael Moore, Gig Harbor, WA (US); Kevin R. Pickup, Paradise, UT (US); Brandon R. Burtenshaw, Nibley, UT (US); DeWayne S. Merritt, Hyrum, UT (US); Collin Vaughn Hodges, West Point, UT (US); Brandon M. Knudsen, Wellsville, UT (US); Eric Davis, Smithfield, UT (US)

(21) Appl. No.: 19/106,403

(22) PCT Filed: Aug. 24, 2023
(86) PCT No.: PCT/US2023/072858

§ 371 Date: Feb. 25, 2025

Related U.S. Application Data
(60) Provisional application No. 63/401,269, filed on Aug. 26, 2022.

(51) Int. Cl. B21D 39/20; B21D 39/04

(52) U.S. Cl. CPC B21D 39/20; B21D 39/046

(57) ABSTRACT

Various embodiments of a compression collar expander assembly for expanding a tubular compression collar are described. For example, the compression collar expander assembly can include a guide plate, a spindle plate, and a plurality of carriages movably disposed on a top surface of the guide plate. Each of the plurality of carriages can include a body, a guide projecting from the body, and a die set comprising a plurality of dies. Each die can include a base coupled to a corresponding one of the plurality of carriages and a prong upstanding from the base. In some embodiments, a tapered expander is configured to assist with expanding the plurality of dies.

### CROSS-REFERENCE TO RELATED APPLICATIONS

The current application claims priority to U.S. Provisional Application No. 63/401,269, filed Aug. 26, 2022, and entitled “Compression Collar Expander Assembly and Method of Use,” which is incorporated by reference herein in its entirety.

### BACKGROUND OF THE DISCLOSURE

### 1. the Field of the Disclosure

The present disclosure relates to compression collar expander assemblies used for expanding a tubular compression collar for coupling a tube to a tube fitting and related methods of use. The compression collars can be used in the biopharmaceutical industry for producing sterile and non-sterile fluid couplings.

### 2. The Relevant Technology

Within the biopharmaceutical industry there exists many applications where various fluids are stored, mixed, processed, and transferred, or transported. Such fluids can be expensive and it is often critical that they be maintained sterile. To help maintain sterility and to eliminate the need for cleaning of containers, many fluids are now processed and stored in sterile polymeric bags. To facilitate transfer of fluid between different bags, polymeric tubing or line sets are connected to barbed ports secured to the bags.

One weakness with the traditional friction fit connection between the tubing and the barbed port is that when the fluid system is pressurized, the applied pressure can cause the tubing to separate or lift off of the face of the barb on the port. This separation can cause potential leaks and contamination of the fluid. Another problem exists when customers handle the fluid system and manipulate the barbed port connection. Such handling can again cause the sealed connection between the barbed port and the tubing to be broken, thereby risking contamination of the fluid.

To assist in eliminating the above problems, the biopharmaceutical industry has adopted the use of cable ties, also known as zip ties, which are manually secured around the tubing over each barbed port. The cable ties provide a compressive force on the tubing that produces a sealed engagement between the tubing and the port, even when the system is handled or pressurized.

Although cable ties have been largely found to be effective, such use has its shortcomings. For example, once a cable tie is secured in place, the unused free end is typically cut off. As a result of the cut, however, the remaining free end now has sharp corners that can potentially puncture or otherwise damage the polymeric bags of the fluid system, especially when the polymeric bags are folded over the cable ties for transport or storage. Although bubble wrap or other packing can be placed over each cable tie, such a process is time consuming, labor intensive and subject to error or failure.

Furthermore, a cable tie does not provide a uniform compressive force around the tube to which it is secured. Rather, as a result of the geometry of the cable tie, there is a location at the intersection of where the tape section feeds into the locking head where a gap or at least decreased compressive force is typically formed between the cable tie and tubing. As a result, there is an area of weakness between the tube and barbed port that has a higher probability of leaking and permitting contamination of the fluid. Cable ties are also problematic in that they can be difficult to attach and difficult to control the amount of compressive force they apply. Furthermore, after a cable tie is tensioned, it will naturally relax over time, thereby decreasing compression on the port. Other problems also exist with using conventional cable ties.

In one attempt to resolve the above problems associated with cable ties, annular compression collars have been developed that have resilient, elastic properties. During use, the compression collars are radially expanded, placed over a tube having a tube fitting received therein, and then allowed to constrict. As the compression collar resiliently constricts, it compresses the tube against the tube fitting to form a sealed connection therebetween. One example of such compression collars is disclosed in US Patent Publication No. 2018/0187809, published Jul. 5, 2018.

This application is directed to improved apparatus and method for consistently and uniformly expanding compression collars.

### SUMMARY

Systems and methods are provided for a compression collar expander assembly and methods of expanding a tubular compression collar used in coupling a tube to a tube fitting.

In some embodiments, a compression collar expander assembly for expanding a tubular compression collar used in coupling a tube to a tube fitting can include a guide plate having a top surface and an opposing bottom surface with a plurality of lower guide channels extending therethrough. The compression collar expander assembly can include a spindle plate disposed adjacent to the bottom surface of the guide plate, and the spindle plate can be rotatable relative to the guide plate and can have a top surface with an expansion channel formed thereon. The compression collar expander assembly can include a plurality of carriages movably disposed on the top surface of the guide plate, and the plurality of carriages can include at least three carriages that can be radially spaced about an axis extending through the guide plate and can be radially aligned with the axis. Each of the plurality of carriages can include a body and a guide projecting from the body, through a corresponding one of the lower guide channels formed on the guide plate, and into the expansion channel. The compression collar expander assembly can include a die set including a plurality of dies, and each die can include a base coupled to a corresponding one of the plurality of carriages and a prong upstanding from the base.

In some embodiments, the expansion channel can have a spiral configuration that encircles the axis. Each prong can have a wedge-shaped configuration with opposing side faces that can extend between an inside face and an opposing outside face, and the outside face can be formed with a curvature. The curvature of the outside face can be an arc of a circle. The opposing side faces can converge toward the inside face and can diverge toward the outside face. The plurality of dies can include at least 3, 5, 7, 9, 11, or 13 separate dies. Each of the plurality of dies can be separate from each other, can be radially spaced about the axis, and can be radially aligned with the axis. Each of the plurality of carriages can have a mounting hole formed thereon or a mounting post projecting therefrom, each of the plurality of dies can have the other of the mounting hole or mounting post formed thereon, and each mounting post can be received with a corresponding mounting hole so as to couple each die to a corresponding carriage. Each carriage can include a plurality of travel wheels rotatably mounted to the body and resting on the top surface of the guide plate. The guide can include a projection extending from the body and extending into the corresponding lower guide channel, and a lower guide wheel rotatably mounted to the projection and at least partially disposed within the expansion channel. The guide can include an axle projecting from the body and a lower guide wheel rotatably mounted to the axle and at least partially disposed within the expansion channel. The plurality of carriages can include at least 5, 7, 9, 11, or 13 separate carriages. The compression collar expander assembly can include a cover plate secured to the guide plate so that the plurality of carriages are disposed between the guide plate and the cover plate. The cover plate can have a top surface and an opposing bottom surface with a plurality of upper guide channels being formed on the bottom surface, and each carriage can have an upper guide wheel rotatably mounted to the body and disposed within a corresponding one of the plurality of upper guide channels. The compression collar expander assembly can include a motor coupled with the spindle plate, and the motor can be configured to rotate the spindle plate in opposite directions. The motor can be a servo motor. Rotation of the spindle plate in opposite directions can cause each of the plurality of carriages to move between an expanded position wherein each of the plurality of carriages are moved radially outward away from the axis and a contracted position wherein each of the plurality of carriages are moved radially inward toward the axis.

In an interrelated aspect, a compression collar expander assembly for expanding a tubular compression collar used in coupling a tube to a tube fitting can include a housing bounding a compartment, an expansion mechanism disposed within the compartment of the housing, and a die set comprising a plurality of dies. The expansion mechanism can include a guide plate having a top surface and an opposing bottom surface and a plurality of carriages disposed on the top surface of the guide plate. The plurality of carriages can include at least three carriages that can be radially spaced about an axis extending through the guide plate and can be radially aligned with the axis. Each of the plurality of carriages can be movable between an expanded position wherein each of the plurality of carriages are moved radially outward away from the axis and a contracted position wherein each of the plurality of carriages are moved radially inward toward the axis. Each die can comprise a base coupled to a corresponding one of the plurality of carriages and a prong upstanding from the base.

In some embodiments, the housing can have an access opening formed thereon that can communicate with the compartment, and the die set can be removable from the compartment of the housing through the access opening. The compression collar expander assembly can include a cover that can be movable between a first position wherein the cover blocks the access opening and a second position wherein the access opening is unblocked. The housing can include a front panel, a back panel. and opposing side panels that each extend to a top panel, and the access opening can extend through the top panel. The access opening can be circular and can have a maximum diameter that is less than 22 cm, 18 cm, or 14 cm. A collection opening can be formed on the housing, a collection bin can be removably disposed within the compartment of the housing, and a collection tube can be aligned between the collection opening and the collection bin so that when a defective compression collar is passed through the collection opening, the compression collar will travel down the collection tube and deposit into the collection bin. The compression collar expander assembly can include a sensor disposed within the housing, and the sensor can be configured to detect when the defective compression collar is passed down through the collection tube. The guide plate can have a plurality of lower guide channels extending therethrough, the compression collar expander assembly can include a spindle plate disposed adjacent to the bottom surface of the guide plate, and the spindle plate can be rotatable relative to the guide plate and having a top surface with an expansion channel formed thereon. The expansion channel can have a spiral configuration. Each carriage can include a body and a guide projecting from the body, through a corresponding one of the lower guide channels formed on the guide plate, and into the expansion channel. The compression collar expander assembly can include a motor coupled with the spindle plate, and the motor can be configured to rotate the spindle plate in opposite directions. Each prong can have a wedge-shaped configuration with opposing side faces that can extend between an inside face and an opposing outside face, and the outside face can be formed with a curvature. Each carriage can include a body and a plurality of travel wheels rotatably mounted to the body and resting on the top surface of the guide plate. The plurality of carriages can include at least 5, 7, 9, 11 or 13 separate carriages.

In an interrelated aspect, a compression collar expander system for expanding a tubular compression collar used in coupling a tube to a tube fitting can include a compression collar expander assembly and a plurality of separate die sets. The compression collar expander assembly can include a housing bounding a compartment and an expansion mechanism disposed within the compartment of the housing. The expansion mechanism can include a guide plate having a top surface and an opposing bottom surface, and a plurality of carriages disposed on the top surface of the guide plate. The plurality of carriages can include at least three carriages that can be radially spaced about an axis extending through the guide plate and can be radially aligned with the axis. Each of the plurality of carriages can be moveable between an expanded position wherein each of the plurality of carriages are moved radially outward away from the axis and a contracted position wherein each of the plurality of carriages are moved radially inward toward the axis. The plurality of separate die sets can each have a different configuration and can each be configured to removably couple with the expansion mechanism, each die set can include a plurality of dies, and each die can include a base for coupling to a corresponding one of the plurality of carriages and a prong upstanding from the base.

In some embodiments, the prongs for any of the plurality of die sets each can have the same configuration. The prongs for any one of the plurality of die sets can have a different configuration than the prongs of any of the other die sets. Each prong can have a wedge-shaped configuration with opposing side faces that can extend between an inside face and an opposing outside face, and the outside face can be formed with a curvature. The plurality of dies for each die set can include at least 5, 7, 9, 11 or 13 separate dies. Each of the plurality of carriages can have a mounting hole formed thereon or a mounting post projecting therefrom, each plurality of dies can have the other of the mounting hole or mounting post formed thereon, and each mounting post can be received with a corresponding mounting hole so as to couple each die to a corresponding carriage. Each of the plurality of die sets can be sized to pass through an access opening formed on the housing for coupling with the expansion mechanism. The compression collar expander system can include a cover that can be movable between a first position wherein the cover blocks the access opening and a second position wherein the access opening is unblocked. The guide plate can have a plurality of lower guide channels extending therethrough, the compression collar expander system can include a spindle plate disposed adjacent to the bottom surface of the guide plate, and the spindle plate can be rotatable relative to the guide plate and can have a top surface with an expansion channel formed thereon. Each carriage can include a body and a guide projecting from the body, through a corresponding one of the lower guide channels formed on the guide plate, and into the expansion channel.

In an interrelated aspect, a die set for use in expanding a tubular compression collar used in coupling a tube to a tube fitting can include a plurality of dies, and each die can include an elongated base that can have a top surface and an opposing bottom surface that can extend between a first end and an opposing second end, a prong that can upstand from the top surface of the base at the first end and can have a wedge-shaped configuration with opposing side faces that can extend between an inside face and an opposing outside face, the outside face can be formed with a curvature, and a leg that can downwardly project from the bottom surface of the base, and the leg can have an inside face and an opposing outside face.

In some embodiments, at least a portion of the outside face of the leg can be planar. The inside face of the leg can be offset from the inside face of the prong. The die set can include a mounting hole that can extend through the base between the top surface and the opposing bottom surface. The die set can include a placement hole that can extend through the base between the top surface and the opposing bottom surface and can be spaced apart from the mounting hole. The curvature of the outside face of the prong can be an arc of a circle. The opposing side faces of the prong can converge toward the inside face and can diverge toward the outside face. The plurality of dies can include at least 5, 7, 9, 11 or 13 separate dies. Each of the plurality of dies can be separate from each other, can be radially spaced about an axis, and can be radially aligned with the axis.

In an interrelated aspect, a method for expanding a tubular compression collar used in coupling a tube to a tube fitting can include positioning the tubular compression collar on a die head of a die set so that the compression collar encircles the die head, the die head can be comprised of a plurality of separate prongs radially spaced about an axis, and radially displacing a plurality of carriages that can be radially spaced about the axis so that the plurality of carriages can move radially outward away from the axis, each of the plurality of carriages can be coupled to a corresponding one of the plurality of prongs, and the radial displacement of the plurality of carriages can cause radial displacement of each of the plurality of prongs which in turn can radially outwardly expand the tubular compression collar encircling the die head.

In some embodiments, radially displacing the plurality of carriages can include rotating a spindle plate about an axis of rotation, the spindle plate can have a top surface with an expansion channel formed thereon, and the rotation of the spindle plate can cause the plurality of carriages to be displaced radially outward away from the axis. Rotating the spindle plate can include activating a motor coupled with the spindle plate, and the motor can be configured to rotate the spindle plate. Each carriage can include a body having a plurality of travel wheels rotatably mounted thereto. The plurality of carriages can include at least 5, 7, 9, 11 or 13 separate carriages.

In an interrelated aspect, a method for expanding a tubular compression collar used for coupling a tube to a tube fitting can include selecting a die set from a plurality of die sets where each can have a different configuration, each of the plurality of die sets can include a plurality of dies that are separate from each other, each of the plurality of dies can include a base and a prong upstanding from the base, the prongs can be grouped together to form a die head; coupling the selected die set to an expansion mechanism disposed within a compartment of a housing; fitting a tubular compression collar on the die head of the selected die set coupled to the expansion mechanism so that the tubular compression collar encircles the die head; and activating the expansion mechanism so that expansion mechanism causes the selected die head to radially outwardly expand the tubular compression collar.

In some embodiments, the method for expanding a tubular compression collar used for coupling a tube to a tube fitting can include moving a cover so as to unblock an access opening formed on the housing, where the access opening can communicate with the compartment of the housing, and passing the selected die set through the unblocked access opening on the housing for coupling the selected die set to an expansion mechanism. The method for expanding a tubular compression collar used for coupling a tube to a tube fitting can include moving the cover back to block the access opening prior to activating the expansion mechanism. The plurality of die sets can include at least 3, 5, 7, 9, 11, or 13 die sets where each can have a different configuration.

In an interrelated aspect, a method for expanding a tubular compression collar used for coupling a tube to a tube fitting can include positioning an annular first calibration ring around a plurality of prongs that can form a portion of a die set disposed on a compression collar expander assembly, activating the compression collar expander assembly so that the plurality of prongs are radially outwardly displaced so as to bias against an interior surface of the first calibration ring, removing the first calibration ring from around the plurality of prongs, fitting a tubular compression collar around the plurality of prongs, and activating the compression collar expander assembly so that the plurality of prongs are displaced radially outward away from a common axis, where the prongs can radially outwardly expand the tubular compression collar, where the compression collar expander can measure a force being applied by the plurality of prongs to the first calibration ring, and determine whether the measured force is within a predetermined tolerance of a first predetermined force.

In some embodiments, the first calibration ring can include metal. The first calibration ring can be not radially outwardly expanded by the plurality of prongs.

In an interrelated aspect, a compression collar expander assembly for expanding a tubular compression collar used in coupling a tube to a tube fitting can include a tapered expander and a die set. The tapered expander can include an outer surface that increases in cross sectional diameter between a first end and a second end of the tapered expander. The die set can include a plurality of dies, each die can include a base and a prong upstanding from the base, and each die can include a tapered inside face extending along the prong that slidably mates with the outer surface of the tapered expander.

In some embodiments, the tapered expander can include a conical shape. The compression collar expander assembly can include a compressible material positioned along an outer circumference of the base of each die of the die set, and the compressible material can bias the die set into a compressed configuration. The compressible material can decompress and force the die set into a compressed configuration when the tapered expander is in a first position. The compressible material can be compressible for allowing the die set to form an expanded configuration when the tapered expander is in a second position. Each prong can have a wedge-shaped configuration with opposing side faces that can extend between the inside face and an opposing outside face, and the outside face can be formed with a curvature. The curvature of the outside face can be an arc of a circle. The opposing side faces can converge toward the inside face and can diverge toward the outside face. The inside face can be rounded for conforming to the outer surface of the tapered expander. The compression collar expander assembly can include a motor coupled with the tapered expander, and the motor can be configured to linearly translate the tapered expander along a longitudinal axis between the first position and the second position. Linear translation of the tapered expander into the second position can cause the tapered expander to apply an increased radial force against the inside face to thereby cause the die set to radially expand. Linear translation of the tapered expander into the first position can cause the tapered expander to apply a decreased radial force against the inside face to thereby cause the die set to radially contract.

### BRIEF DESCRIPTION OF THE DRAWINGS

Various embodiments of the present disclosure will now be discussed with reference to the appended drawings. It is appreciated that these drawings depict only typical embodiments of the disclosure and are therefore not to be considered limiting of its scope.

FIG. 1 is a perspective view of a compression collar expander assembly;

FIG. 2 is a perspective view of the expander assembly shown in FIG. 1 in a partially disassembled state;

FIG. 3 is a schematic view of a CPU and memory used in the expander assembly shown in FIG. 1;

FIG. 4 is a top perspective view of the expander assembly shown in FIG. 1 showing an internal cover and related rails;

FIG. 5 is a perspective view of the expander assembly shown in FIG. 1 showing an internal expander mechanism;

FIG. 6 is an enlarged perspective view of the expander mechanism shown in FIG. 5 having a die set mounted thereon;

FIG. 7A is an enlarged top perspective view of the die set shown in FIG. 6;

FIG. 7B is a bottom perspective view of the die set shown in FIG. 7A;

FIG. 8 is a perspective view of one die of the die set shown in FIG. 7A;

FIG. 9 is a front perspective view of the expander mechanism shown in FIG. 6;

FIG. 10 is a cross sectional side view of the expander mechanism shown in FIG. 9;

FIG. 11 is a top perspective view of the spindle plate of the expander mechanism shown in FIG. 10;

FIG. 12 is a top perspective view of a guide plate of the expander mechanism shown in FIG. 10;

FIG. 13 is a top perspective view of a channel plate of the expander mechanism shown in FIG. 10;

FIG. 14 is a top perspective view of the expander mechanism shown in FIG. 9 showing a plurality of carriages mounted on the guide plate;

FIG. 15 is an enlarged perspective view of one of the carriages shown in FIG. 14;

FIG. 16 is a bottom perspective view of a cover plate of the expander mechanism shown in FIG. 9;

FIG. 17 is a perspective view of a carriage shown in FIG. 15 being disposed on the guide plate shown in FIG. 12;

FIG. 18 is a bottom perspective view of the carriage and guide plate shown in FIG. 17;

FIG. 19 a top perspective view of the expander mechanism shown in FIG. 9 in a partially disassembled state;

FIG. 20 is a perspective view of a die of the die set shown in FIG. 7A mounted on the carriage shown in FIG. 15;

FIG. 21 is a perspective view of a compression collar mounted on the die set of FIG. 6 with the die set being in a contracted state;

FIG. 22 is a perspective view of the die set shown in FIG. 21 moved to an expanded state so as to expand the compression collar;

FIG. 23 is a perspective view of the compression collar shown in FIG. 21;

FIG. 24 is a perspective view of the compression collar in the expanded state shown in FIG. 22 being disposed on a tube aligned with a tube fitting;

FIG. 25 is an elevated side view of the assembled compression collar, tube, and tube fitting shown in FIG. 24 with the compression collar in a contracted state;

FIG. 26 is a cross sectional side view of the assembly shown in FIG. 25;

FIG. 27 is a perspective view of an alternative embodiment of a compression collar shown in FIG. 23 having a single tab;

FIG. 28 is a perspective view of an alternative embodiment of a compression collar shown in FIG. 23 having three tabs;

FIG. 29 is a perspective view of a plurality of die set assemblies each having a die set with a different size/configuration for use with the expander mechanism shown in FIG. 6;

FIG. 30 is a flow chart outlining one method of verifying calibration of the expander assembly using the calibration rings shown in FIG. 29;

FIG. 31A is a front perspective view of an alternative calibration ring;

FIG. 31B is a rear perspective view of the calibration ring shown in FIG. 31A

FIG. 32 is a flow chart outlining an alternative method of calibrating expander assembly 10 using the calibration ring shown in FIGS. 31A and 31B;

FIG. 33 is a flow chart outlining one method of operating the expander assembly shown in FIG. 1 for use in expanding the compression collar shown in FIG. 23;

FIG. 34 is a perspective view of a placement tool which can be used for handling and moving the dies of the die set shown in FIG. 7A;

FIG. 35A is a perspective exploded view of an alternate embodiment of the expansion mechanism including a tapered expander;

FIG. 35B is a side view of the expansion mechanism shown in FIG. 35A;

FIG. 35C is a side section view of the expansion mechanism shown in FIG. 35A taken across section line 35C shown in FIG. 35B and showing the tapered expander in a first position,

FIG. 35D is a side section view of the expansion mechanism shown in FIG. 35A taken across the section line 35C shown in FIG. 35B showing the tapered expander in a second position;

FIG. 35E is a top view of the expansion mechanism shown in FIG. 35A showing an embodiment of the die head in a compressed configuration; and

FIG. 35F is a top view of the expansion mechanism shown in FIG. 35A showing the die head in an expanded configuration.

### DETAILED DESCRIPTION OF THE PREFERRED EMBODIMENTS

Before describing the present disclosure in detail, it is to be understood that this disclosure is not limited to particularly exemplified apparatus, systems, methods, or process parameters that may, of course, vary. It is also to be understood that the terminology used herein is only for the purpose of describing particular exemplary embodiments of the present disclosure and is not intended to limit the scope of the disclosure in any manner.

All publications, patents, and patent applications cited herein, whether supra or infra, are hereby incorporated by reference in their entirety to the same extent as if each individual publication, patent, or patent application was specifically and individually indicated to be incorporated by reference.

The term “comprising” which is synonymous with “including,” “containing,” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, reference to a “slot”includes one, two, or more slots.

As used in the specification and appended claims, directional terms, such as “top,” “bottom,” “left,” “right,” “up,” “down,” “upper,” “lower,” “proximal,” “distal” and the like are used herein solely to indicate relative directions and are not otherwise intended to limit the scope of the disclosure or claims.

Where possible, like numbering of elements have been used in various figures. Furthermore, multiple instances of an element and or sub-elements of a parent element may each include separate letters appended to the element number. For example, two instances of a particular element “10” may be labeled as “10A” and “10B”. In that case, the element label may be used without an appended letter (e.g., “10”) to generally refer to all instances of the element or any one of the elements. Element labels including an appended letter (e.g., “10A”) can be used to refer to a specific instance of the element or to distinguish or draw attention to multiple uses of the element. Furthermore, an element label with an appended letter can be used to designate an alternative design, structure, function, implementation, and/or embodiment of an element. For example, two alternative exemplary embodiments of a particular element may be labeled as “10A” and “10B”. In that case, the element label may be used without an appended letter (e.g., “10”) to generally refer to all instances of the alternative embodiments or any one of the alternative embodiments.

Various aspects of the present devices and systems may be illustrated by describing components that are coupled, attached, and/or joined together. As used herein, the terms “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present. Furthermore, as used herein, the terms “connection,” “connected,” and the like do not necessarily imply direct contact between the two or more elements.

Various aspects of the present devices, systems, and methods may be illustrated with reference to one or more exemplary embodiments. As used herein, the terms “exemplary,” “embodiment” and “exemplary embodiment” mean “serving as an example, instance, or illustration,” and should not necessarily be construed as required or as preferred or advantageous over other embodiments disclosed herein.

Unless defined otherwise, all technical and scientific terms used herein have the same meaning as commonly understood by one of ordinary skill in the art to which the present disclosure pertains. Although a number of methods and materials similar or equivalent to those described herein can be used in the practice of the present disclosure, the preferred materials and methods are described herein.

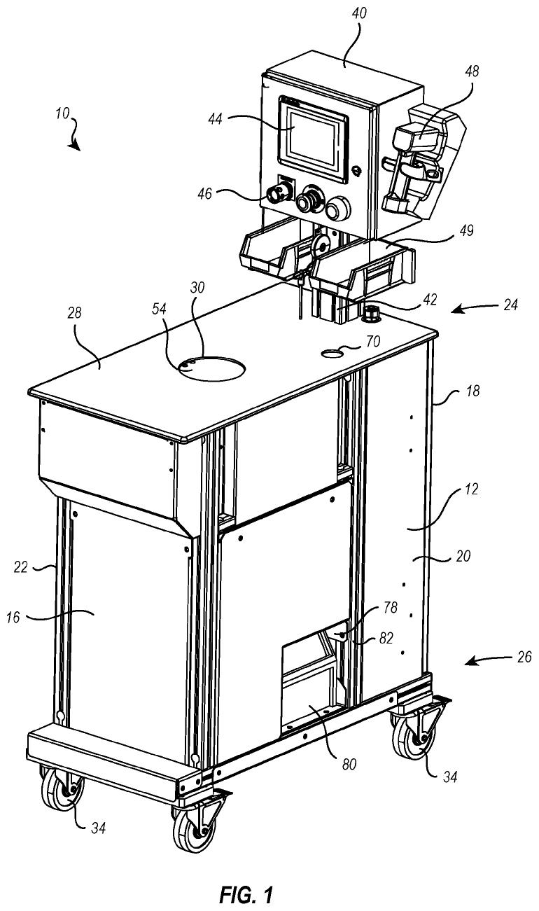

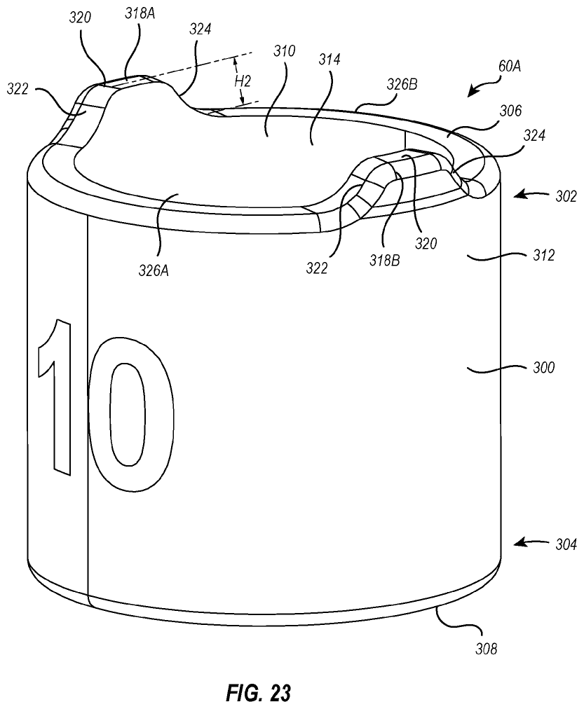

Depicted in FIG. 1 is one exemplary embodiment of a compression collar expander assembly 10 incorporating features of the present disclosure. As will be discussed below in further detail, expander assembly 10 is configured for expanding a tubular compression collar 60A (FIG. 23) used in coupling a tube to a tube fitting. Expander assembly 10 and compression collar 60A can be used in the biopharmaceutical industry for use in facilitating sterile and non-sterile fluid couplings used in the transfer of fluids. For example, compression collars 60A can be used in forming fluid couplings associated with tubing, line sets, fermenters, bioreactors, mixers, storage containers, fluid management systems, and other related equipment. However, expander assembly 10 and compression collar 60A can also be used in other industries for forming sterile and non-sterile fluid couplings, such as in the production, distribution, and/or use of liquid chemicals, food products, or other desired liquids.

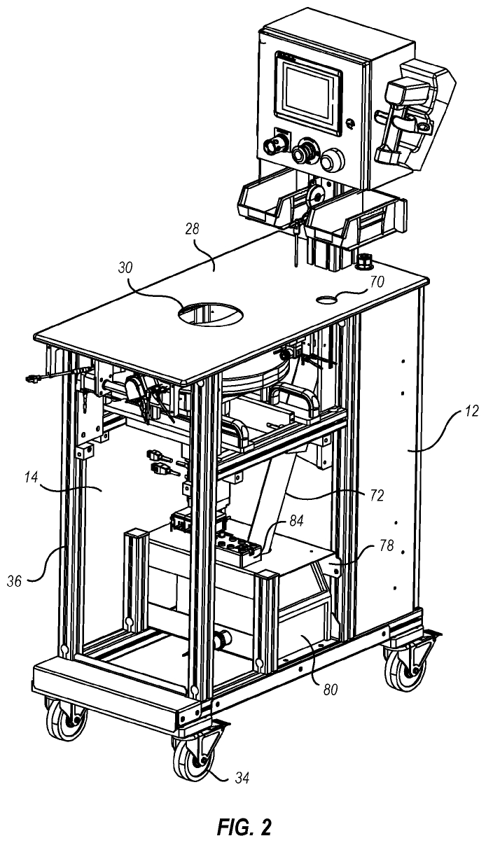

With reference to FIGS. 1 and 2, expander assembly 10 includes a housing 12 bounding a compartment 14. Although housing 12 can has a variety of different configurations, in the depicted embodiment housing 12 has a generally box shaped configuration that includes a front panel 16 and an opposing back panel 18 with opposing side panels 20 and 22 extending therebetween. Panels 16-22 extend between an upper end 24 and an opposing lower end 26. Disposed at upper end 24 is a top panel 28 having an access opening 30 extending therethrough and communicating with compartment 14. Top panel 28 is typically disposed horizontally so as to function as a tabletop.

In the depicted embodiment, access opening 30 is circular. However, in alternative embodiments, access opening 30 can have other shapes such as polygonal, oval, elliptical, or have an irregular configuration. As will be discussed below in further detail, access opening 30 is sized so that an operator can reach through access opening 30 to access compartment 14. In one embodiment, access opening 30 has a diameter of at least 10, 14, 18, or 22 centimeters (cm). Other dimensions can also be used. In other embodiments, access opening 30 can be formed at upper end 24 of one of panels 18-22 so as to communicate with compartment 14.

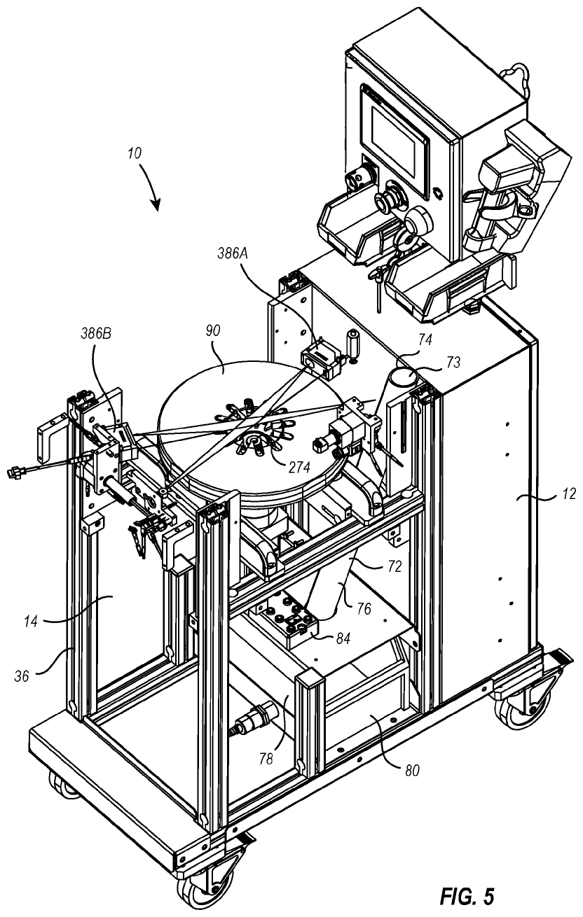

Also extending through top panel 28 is a collection opening 70. As depicted in FIGS. 2 and 5, a collection tube 72 bounding a passage 73 is disposed within compartment 14 of housing 12 and has an upper end 74 that aligns with collection opening 70 and an opposing lower end 76 that communicates with a collection chamber 78 disposed within compartment 14. Removably disposed within collection chamber 78 is a scrap bin 80. Scrap bin 80 can be selectively inserted into and removed from collection chamber 78 through a side opening 82 formed on side panel 20 (FIG. 1) so as to communicate with collection chamber 78. As will be discussed below in greater detail, collection opening 70, collection tube 72 and scrap bin 80 can be used for easily and efficiently collecting and accounting for compression collars 60A that deemed defective or fail to satisfy compliance requirements during the expansion process. Accordingly, collection opening 70 and collection tube 72 are sized so that compression collars 60A can pass therethrough. Collection opening 70 is typically circular but can have other shapes such as polygonal, oval, elliptical, or have an irregular configuration. In one embodiment, collection opening 70 has a diameter that is smaller than the diameter of access opening 30 but is at least or less than 2, 3, 5, 8, 10 or 12 centimeters (cm). A sensor 84, such as an optical sensor, can be positioned adjacent to collection tube 72 and/or collection chamber 78 for detecting when a compression collar 60A has passed down collection tube 72 and into scrap bin 80. Because collection opening 70, collection tube 72, scrap bin 80 and/or sensor 84 relate to safety and/or compliance features, in some embodiments they can be deemed optional and thus eliminated from expander assembly 10.

Returning to FIGS. 1 and 2, disposed at lower end 26 of housing 12 are a plurality of wheels 34 on which housing 12 is supported. In the depicted embodiment, four wheels 34 are used. In other embodiments, expander assembly 10 can be formed with two, three or five or more wheels 34. Wheels 34 can be used to enable easy and selective movement of expander assembly 10/housing 12. In one embodiment, housing 12 combines with wheels 34 to form a rollable cart. In alternative embodiments, wheels 34 can be eliminated.

Housing 12 also includes a frame 36 on which the previously discussed panels can be mounted. It is appreciated that each of the panels 16, 18, 20, 22, and 28 can each comprise a single, unitary panel or can comprise a plurality of subpanels that are coupled together or are adjacently disposed. Panels 16, 18, 20, 22, and 28 can be secured to frame 36 by welding, adhesive or using conventional fasteners.

Expander assembly 10 also includes a control panel 40 coupled to housing 12. In the depicted embodiment, control panel 40 is mounted on a stand 42 that upwardly extends from top panel 28 and is typically secured to frame 36. In alternative embodiments, control panel 40 can be mounted on or incorporated into any of panels 16, 18, 20, or 22 or otherwise be supported on frame 36 so as to be accessible by an operator. Control panel 40 can include a display screen 44, which can be a touch screen, and a plurality of control switches 46. Display screen 44 and control switches 46 can be used in the activation and operation of expander assembly 10. Optionally mounted on control panel 40 is scanner 48. In the depicted embodiment, scanner 48 is a removable, hand-held scanner that can be manually manipulated. In other embodiments, scanner 48 can be securely fixed to control panel 40 or otherwise mounted on housing 12. Optionally mounted on stand 42 and/or control panel 40 are one or more receptacles 49, wherein a plurality of compression collars 60A can be stored for easy access and use.

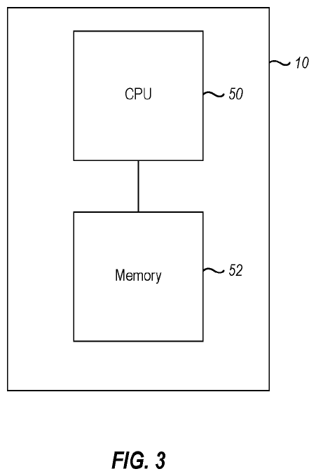

Turing to FIG. 3, expander assembly 10 can also include a central processing unit (CPU) 50 and memory 52. CPU 50 is programmable for operation of expander assembly 10, as will be discussed below, while memory 52 can be any form of computer readable memory that can be accessed by CPU 50, such as non-transitory memory. In one embodiment, CPU 50 and memory 52 can be housed within control panel 40 and/or housing 12 and be controlled through control panel 40 (shown in FIG. 1).

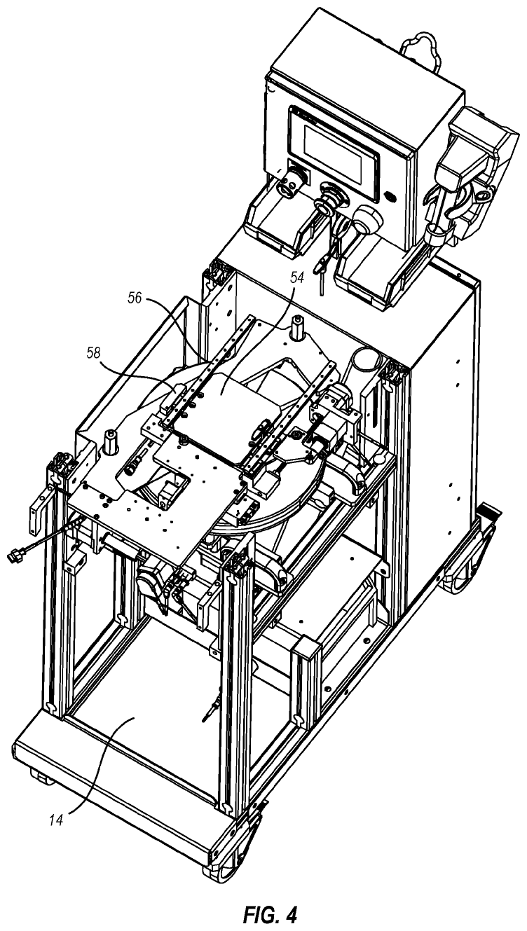

Returning to FIG. 1, in one exemplary embodiment a cover 54 can be used to selectively block and unblock access opening 30. Specifically, FIG. 1 shows cover 54 in a first position where cover 54 blocks access opening 30 so as to prevent an operator from reaching through access opening 30 and accessing compartment 14 (shown in FIG. 2). In FIG. 2, cover 54 is moved to a second position wherein access opening 30 is unblocked so that an operator can freely reach down through access opening 30 to access compartment 14. As depicted in FIG. 4, cover 54 is typically in the form of a plate, panel, or other structure having a planar surface that can cover access opening 30. Cover 54 can be mounted on rails 56 that enable cover 54 to slide back and forth between the first and second positions. A drive mechanism 58, in electrical communication with CPU 50 (shown in FIG. 3), controls movement of cover 54 between the first and second positions. Drive mechanism 58 can comprise a hydraulic, pneumatic, gear, screw, pully, or any other conventional form of driver. As will be discussed below, cover 54 and related components are safety features for the operator and in select embodiments can be eliminated.

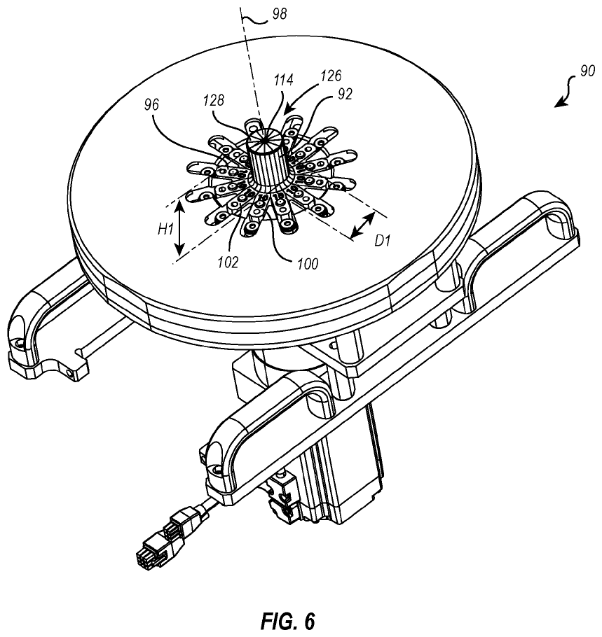

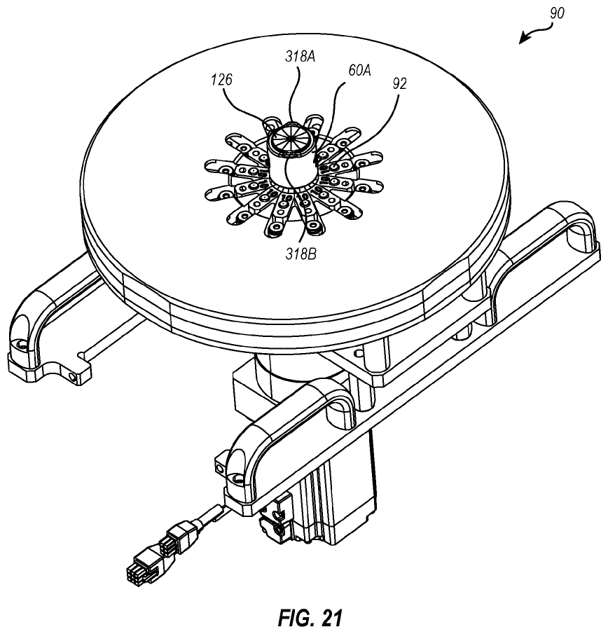

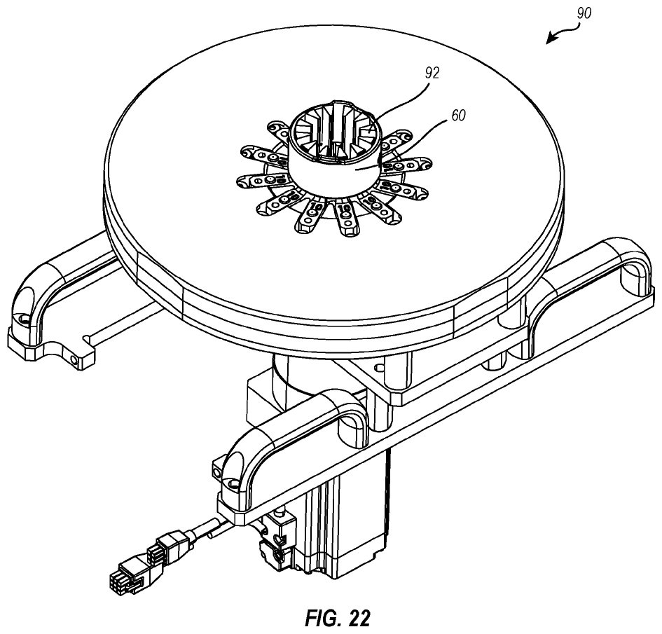

Returning to FIG. 5, expander assembly 10 also includes an expansion mechanism 90 disposed within compartment 14 and secured to frame 36/housing 12. Depicted in FIG. 6 is an enlarged perspective view of expansion mechanism 90 having a die set 92 removeably mounted thereon. As will be discussed below in further detail, the combination of expansion mechanism 90 and die set 92 is used to selectively expand compression collar 60A. For example, as depicted in FIG. 21, compression collar 60A is disposed on die set 92 while die set 92 is in a contracted position. As depicted in FIG. 22, expansion mechanism 90 has been used to radially expand die set 92 to an expanded position which in turn facilitates radial expansion of compression collar 60A. The components and operation of expansion mechanism 90 and die set 92 will now be discussed in greater detail.

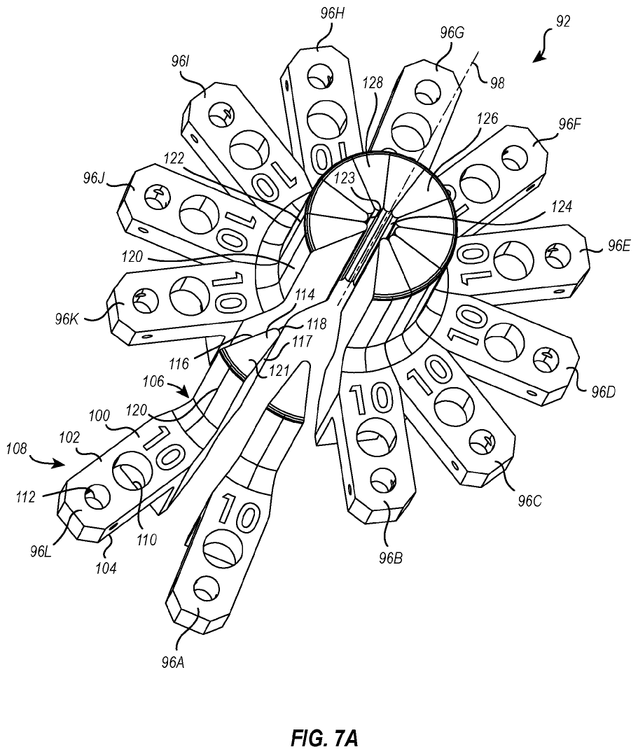

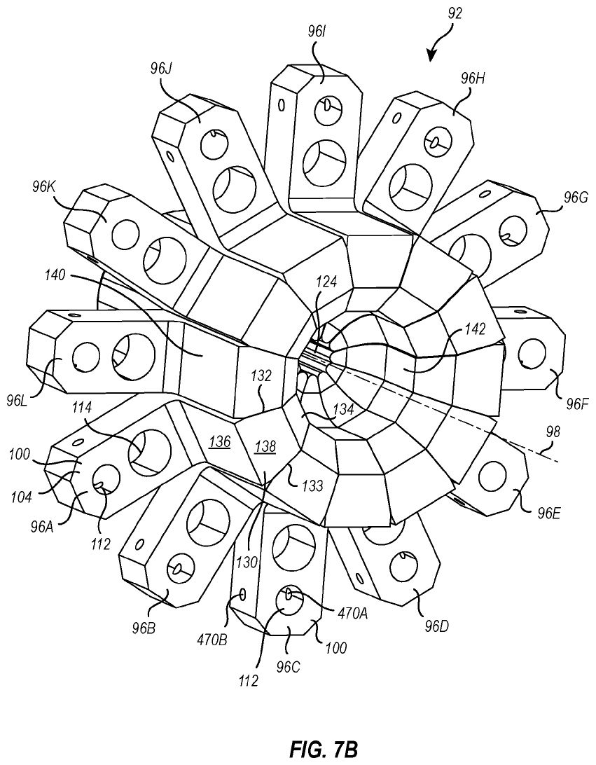

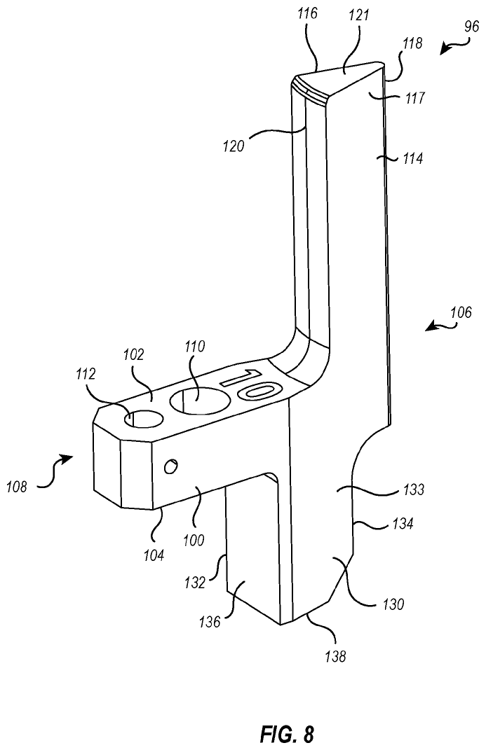

Turning to FIGS. 7A and 7B, die set 92 comprises a plurality of dies 96A-96L that are separate and discrete from each other and that are radially spaced about an axis 98. More specifically, each die 96A-96L is aligned with and radially outwardly projects away from axis 98 at a different angle of orientation so that dies 96A-96L cumulatively encircle or extend about axis 98. In one embodiment, each of dies 96A-96L can have an identical configuration. This enables each of dies 96A-96L to be interchanged with any of the other dies 96A-96L in different orientations while still enabling die set 92 to properly function for its intended purpose. In one embodiment, as depicted in FIGS. 7 and 8, each die 96 can comprise an elongated base 100 having a top surface 102 and an opposing bottom surface 104 that extend between a first end 106 and an opposing second end 108. Extending through base 100 between top surface 102 and bottom surface 104 is a mounting hole 110 and a placement hole 112. As discussed below in greater detail, placement hole 112 is optional and can be used to receive a tool used in the placement and removal of each die 96. In the depicted embodiment, both mounting hole 110 and placement hole 112 are circular with placement hole 112 being smaller than mounting hole 110. In alternative embodiments, mounting hole 110 and placement hole 112 can have the same or different sizes and/or can have other non-circular configurations. However, as will be apparent from the below discussion, the circular configuration of holes 110 and 112 facilitates easy mounting and alignment of die set 92/dies 96.

Upwardly expanding from first end 106 of each base 100 is a prong 114. Each prong 114 is elongated and having a wedge-shaped transverse cross section that includes opposing side faces 116 and 117 that converge toward an inside face 118 and diverge toward an opposing outside face 120. In one embodiment, opposing side faces 116 and 117 are planar, although not required. Inside face 118 can comprise a planar face, a rounded corner, or a sharpened ridge. Outside face 120 typically has the configuration of a smooth, continuous curve that extends or is disposed between side faces 116 and 117. In one exemplary embodiment, outside face 120 forms an arc of a circle. Outside face 120 is the surface that pushes against an interior surface of compression collar 60A during radial expansion of compression collar 60A, as will be discussed further below. Having outside face 120 in the form of a curve, particularly one that is complementary or close to the curvature of the interior surface of compression collar 60A, assists in providing a more uniform force against the interior surface of compression collar 60A so as to provide uniform expansion of compression collar 60A. In contrast, non-curved surfaces that provide a sharp, localized force against compression collar 60A may distort or damage the interior surface of compression collar 60A during expansion. Each of faces 116, 117, 118, and 120 extend upwardly from first end 106 of base 100 to a top face 121. Top face 121 can be planar, as depicted, but it is not required.

When die set 92 is in the contracted position, as shown in FIGS. 6, 7A and 7B, side faces 116 and 117 of each die 96 are adjacently disposed to or are directly abutting against an opposing side face 116 or 117 of an adjacent die 96 so that prongs 114 combine to form a die head 126 having a cylindrical configuration and a circular transverse cross section of which axis 98 is a central axis. Die head 126 has an encircling outside face 122 formed from the combination of outside faces 120 and terminates a top end face 128 formed by the combination of top faces 121. Outside face 122 also has a cylindrical configuration and a circular transverse cross section of which axis 98 is a central axis. Die head 126, when in this contracted state, is configured to receive compression collar 60A thereon (shown in FIG. 21). Although die head 126 is depicted with top end face 128 being flat, top end face 128 can have other configurations. For example, top end face 128 could be rounded, domed, conical, frustoconical, pyramidal, or have other configurations. The tapering of top end face 128 can assist with the alignment and positioning of compression collar 60A on die head 126. As shown in FIG. 7A, die head 126 can also be formed with an inside face 123, formed from the combination of inside faces 118, that encircles or extends about a passage 124 through which axis 98 extends. The formation of passage 124 can help ensure proper alignment and positioning of dies 96 when moved to the contracted state.

Returning to FIG. 6, while in the contracted state, die head 126 has a height HI that extends between top surface 102 of base 100 and top end face 128 and has an outside diameter D1. Depending on the size or sizes of compression collars 60A to be expanded, die set 92 or a plurality of die sets 92 can be produced having height H1 and diameter D1 with variety of different dimension. Commonly, height H1 increases as diameter D1 increases. For example, height H1 can be at least or less than 2 mm, 3 mm, 4 mm, 6 mm, 8 mm, 10 mm, 15 mm, 20 mm, 25 mm, 30 mm, 40 mm, 50 mm, 60 mm, 70 mm or 90 mm or be in a range between any two of the forgoing. Likewise, diameter D1 can be at least or less than 2 mm, 3 mm, 4 mm, 6 mm, 8 mm, 10 mm, 15 mm, 20 mm, 25 mm, 30 mm, 40 mm, 50 mm, 60 mm, 70 mm or 90 mm or be in a range between any two of the foregoing. Other dimensions can also be used. Commonly, although not required, the height H1 is greater than the diameter D1 for each die set 92.

Returning to FIGS. 7B and 8, downwardly projecting from bottom surface 104 of each base 100 is a leg 130. Each leg 130 also has a wedge-shaped transverse cross section with opposing side faces 132 and 133 that converge towards an inside face 134 and diverge toward an opposing outside face 136. Faces 132, 133, 134, and 136 extend to a bottom face 138. Opposing side faces 132 and 133 can also be planar and be disposed adjacent to or abutting against a corresponding opposing side faces 132 or 133 of an adjacent die 96 when die set 92 is in the contracted position. Inside face 134 is typically offset from inside faced 118, i.e., is disposed back toward second end 108. In contrast to outside face 120 of prong 114 which is typically in the form of a curve or arc, outside face 136 of leg 130 is typically planar, for reasons as will be discussed below. When die set 96 is in the contracted position, outside faces 136 again combine to form an encircling outer face 140, typically having a polygonal transverse cross section, while inside faces 134 encircle an opening 142 that communicates with passage 124. Opening 142 is typically larger than passage 124 so as to help maintain a generally uniform thickness for each die 96, thereby helping to ensure uniformity in heat treating of dies 96 which are typically made of metal.

In the depicted embodiment of FIGS. 7B-8, die set 92 is shown being formed of twelve separate dies 96. As a result, the arced/curved outside face 120 of each prong 114 extends over an angle of approximately 30 degrees. In general, increasing the number of dies 96 in die set 92 increases uniform radially distribution of the expansion force that is applied against the interior surface of compression collar 60A as compression collar 60A is radially outwardly expanded by die set 92. However, as the number of dies 96 in die set 92 increases, the thickness of each prong 114 decreases and the surface area of each outside face 120 decreases. Decreasing the thickness of prong 114 too much can result in failure of prong 114 as the expansion force is applied. Furthermore, decreasing the area of outside face 120 too much can result in unwanted localized deformation on the interior surface of compression collar 60A, as discussed above.

As will be discussed further below, die set 92 can be formed in different sizes for use in expanding compression collars of different sizes. In view of the foregoing considerations and other factors, dies sets 92 for expanding smaller compression collars 60A typically include fewer dies 96 while die sets 92 for expanding larger compression collars 60A typically include more dies 92. Accordingly, depending on intended use and to optimize performance, die sets 92 can be formed that include or have at least 2, 3, 4, 5, 6, 8, 10, 12, 14 or 16 separate dies 96 or have a number of dies 96 within a range of any two of the foregoing numbers. For example, it is common to have die sets 92 having between with between 4 and 16 dies 96 or more common between 5 and 14 dies 96. Likewise, outside face 120 of each prong 114 typically extends over an angle of at least 15 degrees, 20 degrees, 25 degrees, 30 degrees, 35 degrees, 40 degrees, 50 degrees, 60 degrees, 65 degrees, 70 degrees or 80 degrees or in a range between any two of the foregoing degrees. For example, outside faces 120 commonly extend over an angle in a range between 20 degrees and 80 degrees with between 25 degrees and 65 degrees being more common. Other values can also be used.

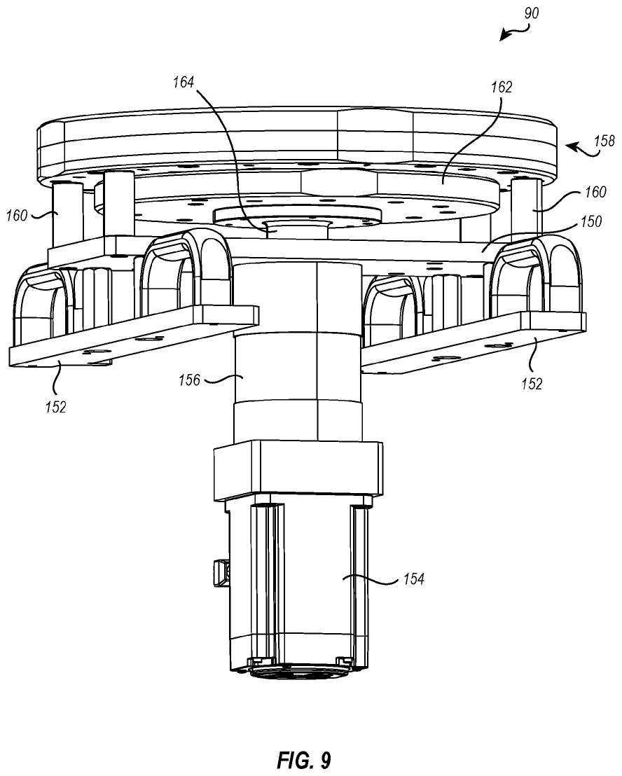

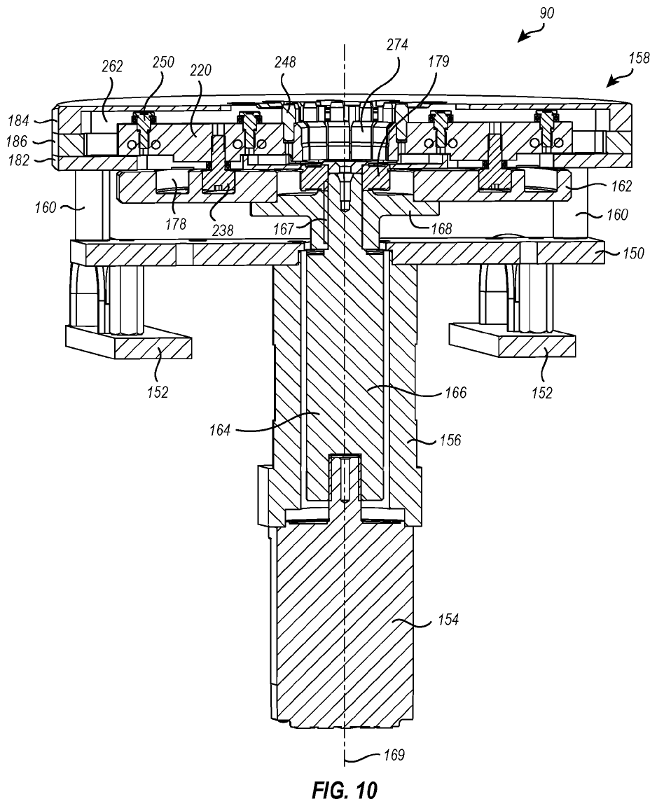

Turning to FIGS. 9 and 10, expansion mechanism 90 comprises a support 150 that is rigidly secured to frame 36/housing 12 (shown in FIG. 2) such as through the use of braces 152. A motor 154 is mounted on support 150 and can be secured thereto by a tubular sleeve 156. As will be discussed further below, motor 154 can comprise any motor that can facilitate rotation of a drive shaft in opposite directions over predefined angles. Motor 154 is typically an electrical motor and is more commonly a servo motor. Expansion mechanism 90 further includes an expander plate assembly 158 disposed above support 150. Expander plate assembly 158 can be secured to support 150 by a plurality of braces 160. Positioned between expander plate assembly 158 and support 150 is a spindle plate 162. Spindle plate 162 is rotatable relative to support 150 and expander plate assembly 158. A drive shaft 164 extends between motor 154 and spindle plate 162 while passing through sleeve 156 so that motor 154 can facilitate selective rotation of spindle plate 162 relative to expander plate assembly 158. Drive shaft 164 rotates about a rotational axis 169. In the depicted embodiment, drive shaft 164 comprises a primary drive shaft 166 extending from motor 154 and passing through sleeve 156 and support 150 and also a flange 168 that couples to primary drive shaft 166, such as through a keyway 167, and connects to spindle plate 162. In alternative embodiments, primary drive shaft 166 and flange 168 can be formed as a single, integral, unitary structure.

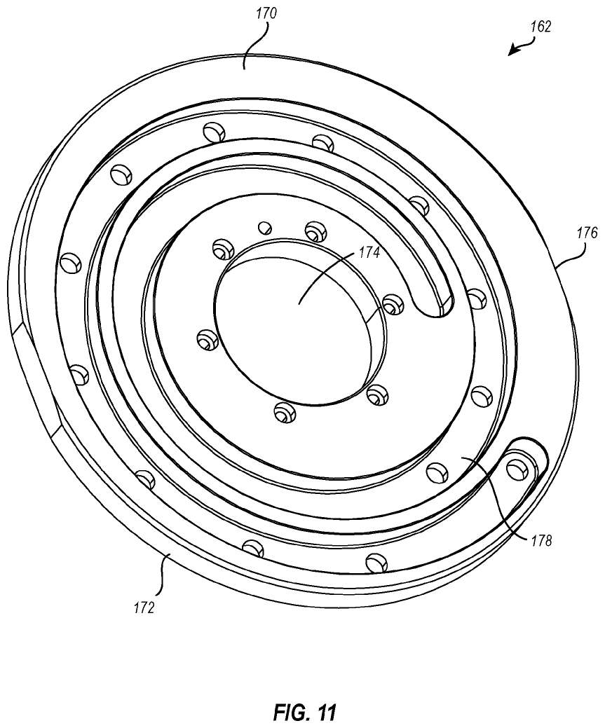

Turning to FIG. 11, spindle plate 162 has a top surface 170 and an opposing bottom surface 172 that each extend between a central opening 174 and an outer perimeter edge 176. Recessed into top surface 170 is an expansion channel 178 which encircles central opening 174 and radially spirals outward as it encircles central opening 174. Expansion channel 178 faces towards expander plate assembly 158. Opening 174 is provided on spindle plate 162 so that a bearing assembly 179 (shown in FIG. 10) can be received therein that extends between the end of drive shaft 164/primary drive shaft 166 and expander plate assembly 158. Bearing assembly 179 provides added stability to drive shaft 164 while still enabling drive shaft 164 to rotate relative to expander plate assembly 158. In other embodiments, opening 174 can be eliminated and the end of drive shaft 164/primary drive shaft 166 can connect directly to spindle plate 162.

Returning to FIG. 10, in general, expander plate assembly 158 comprises a guide plate 182, a cover plate 184, and a channel plate 186 disposed between cover plate 184 and guide plate 182. Guide plate 182, cover plate 184 and channel plate 186 are typically each secured together so as to preclude movement relative to each other. Guide plate 182 is disposed above and directly adjacent to spindle plate 162.

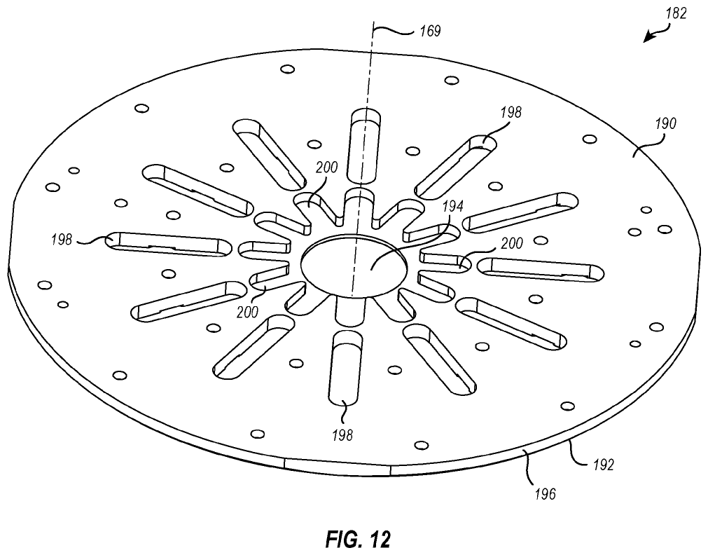

As depicted in FIG. 12, guide plate 182 comprises a top surface 190 and an opposing bottom surface 192 that radially extend between a central opening 194 and an outer perimeter edge 196. Guide plate 182 is mounted to support 150 (shown in FIG. 10) so that rotational axis 169 centrally passes through central opening 194. Extending through guide plate 182 between top surface 190 and bottom surface 192 are a plurality of guide channels 198. Guide channels 198 are linear and elongated and are radially aligned with rotational axis 169 so as to radially outwardly project away from central opening 194 and/or rotational axis 169 at locations spaced apart from central opening 194 and/or rotational axis 169. Guide channels 198 are equally radially spaced apart around central opening 194 and/or rotational axis 169 and the number of guide channels 198 is typically equal to the maximum number of dies 96 that will be used in any die set 92 to be mounted on expansion mechanism. As such, the number of guide channels 198 can be equal to the same number dies 96 that can be used in die set 92, as previously discussed. A plurality of guide paths 200 are recessed into top surface 190 of guide plate 182 so as to each align with a corresponding one of guide channels 198. Guide paths 200 are linear and radially aligned with central opening 194/rotational axis 169 and are disposed between central opening 194 and guide channels 198. In alternative embodiments, guide paths 200 can extend all the way through guide plate 182 and/or guide paths 200 can interconnect with their corresponding guide channel 198. The recessing of guide paths 200 and not directly connecting with guide channels 198 provides improved structural stability to guide plate 182.

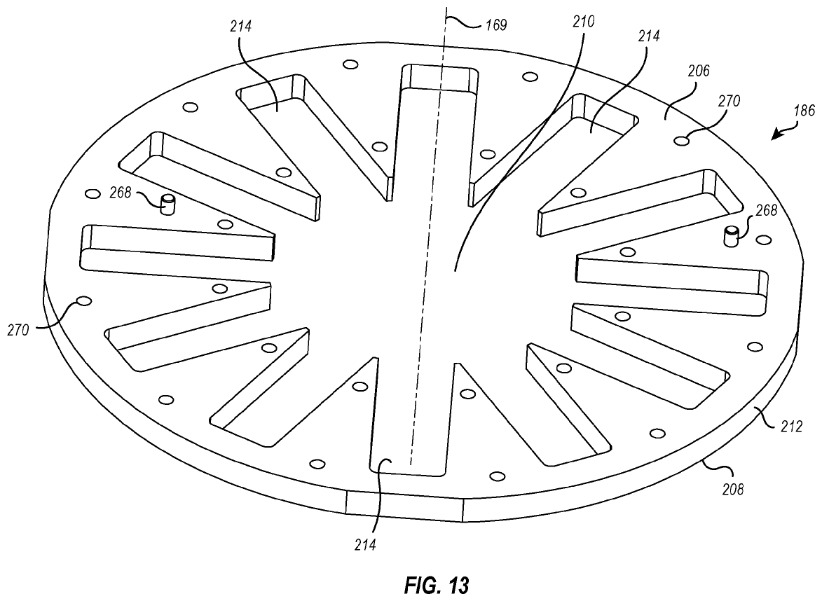

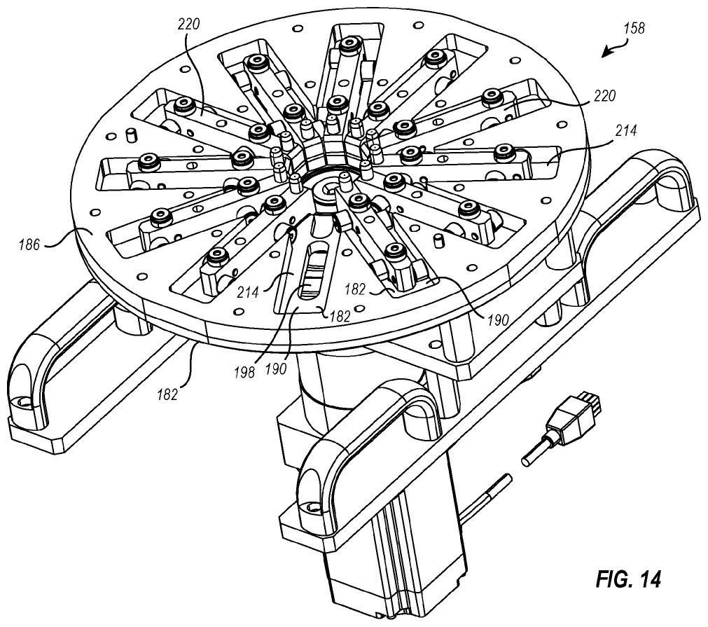

Turning to FIG. 13, channel plate 186 has a top surface 206 and an opposing bottom surface 208 that extend between a central opening 210 and an outer perimeter edge 212. Channel plate 186 mounts on top of guide plate 182 so that rotational axis 169 passes through central opening 210. A plurality of alignment channels 214 extend through channel plate 186 between top surface 206 and bottom surface 208 so to communicate with central opening 210. Alignment channels 214 are elongated and radially aligned with rotational axis 169/central opening 210. Alignment channels 214 are equally radially spaced apart around central opening 210/rotational axis 169 and the number of alignment channels 214 is typically equal to the number of guide channels 198, discussed above. Bottom surface 208 of channel plate 186 sits on top of top surface 190 of guide plate 182 so that each guide channel 198 is centrally aligned with and extends along a corresponding alignment channel 214 (shown in FIG. 14). Alignment channels 214 are typically wider than guide channels 198. For ease of manufacturing, guide plate 182 and channel plate 186 are formed as separate plates that are connected together. In alternative embodiments, however, guide plate 182 and channel plate 186 can be integrally formed as a single, integral, unitary structure, i.e., formed as a single plate as opposed to two plates connected together. Alternatively, cover plate 184 and channel plate 186 can be integrally formed as a single, integral, unitary structure.

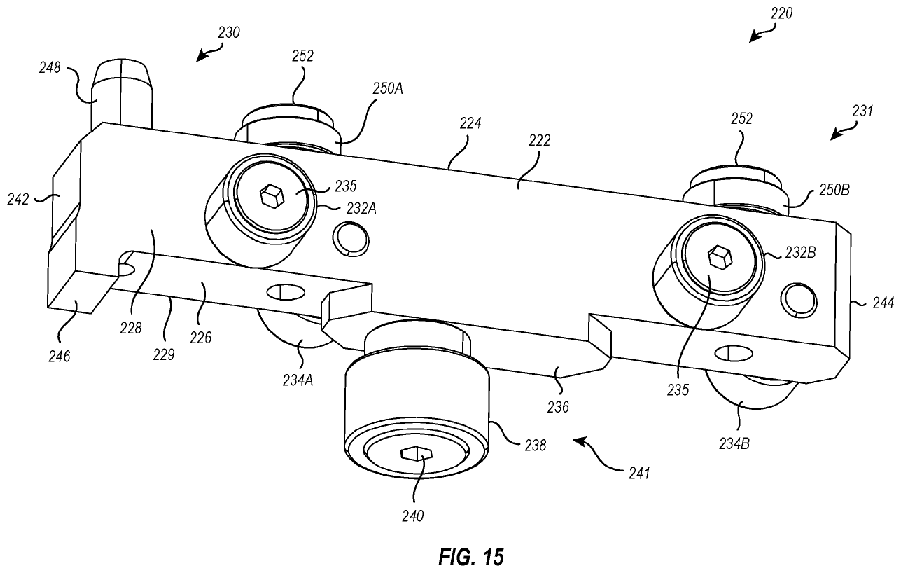

Turning to FIG. 14, channel plate 186 is shown disposed on guide plate 182. As further shown therein, expander plate assembly 158 also comprises a plurality of carriages 220. The number of carriages 220 is typically equal to the number of alignment channels 214/guide channels 198. Each carriage 220 is received within a corresponding alignment channel 214 so as to rest on top surface 190 of guide plate 182. For purposes of clarity, one of carriages 220 is hidden in FIG. 14. As depicted in FIG. 15, each carriage 220 comprises an elongated body 222 having a top surface 224, an opposing bottom surface 226, and opposing side surfaces 228 and 229 that each longitudinally extend between a first end 230 and an opposing second end 231. First end 230 terminates at a fist end face 242 while second end 231 terminates at a second end face 244. Downwardly projecting from bottom surface 226 is a nose 246. Although not required, in one embodiment nose 246 can also extend from first end face 242. Upwardly projecting from top surface 224 is a mounting pin 248. As will be discussed below, mounting pin 248 is configured to be received within mounting hole 110 of a die 96 of die set 92 (shown in FIG. 7A). As such, mounting pin 248 typically has a configuration complementary to the configuration of mounting hole 110, as previously discussed, so that mounting pin 248 can easily slide into mounting hole 110. In the depicted embodiment, mounting pin 248 has a circular transverse cross section with a cylindrical configuration.

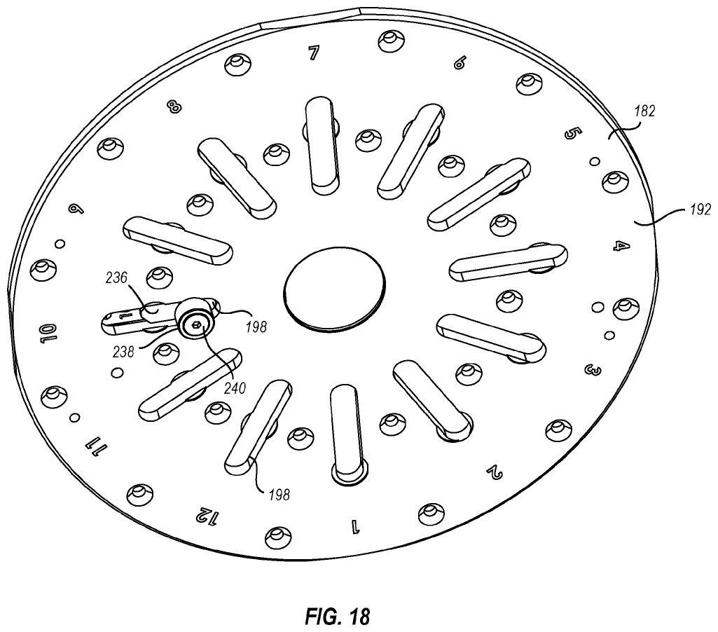

Rotatably mounted on side surface 228 are a pair of spaced apart travel wheels 232A and 232B while rotatably mounted on side surface 229 are a pair of spaced apart travel wheels 234A and 234B. For example, in one embodiment, axles 235 outwardly project from side surfaces 228 and 229, such as orthogonally, and travel wheels 232 and 234 are rotatably mounted thereon. Travel wheels 232 and 234 extend below bottom surface 226 of body 222 so that travel wheels 232 and 234 can directly ride on and rotate over top surface 190 of guide plate 182 (shown in FIG. 14). A projection 236 centrally projects down from bottom surface 226 of body 222 and is slidably received within guide channel 198 of guide plate 182 (shown in FIG. 18). Projection 236 has a length shorter than a length of guide channel 198 so as to enable carriage 220 to travel back and forth on guide plate 182 while projection 236 slides within guide channel 198.

Downwardly extending from projection 236 is a rotatable lower guide wheel 238. Specifically, an axle 240 projects down from projection 236 with lower guide wheel 238 being rotatably disposed thereon. In this orientation, axle 240 can project orthogonal to axles 235 and lower guide wheel 238 can project orthogonal to and rotate orthogonal to travel wheels 232 and 234. As discussed below, lower guide wheel 238 is configured to be received within and travel along expansion channel 178 of spindle plate 162 (shown in FIG. 11). Projection 236, axle 240 and lower guide wheel 238 combine to form a guide 241. In an alternative embodiment, projection 236 can be eliminated so that guide 241 only includes axle 240 and lower guide wheel 238. In this embodiment, axle 240 projects down directly from bottom surface 226 of body 222 and is slidably received within and passes through guide channel 198 of guide plate 182 (shown in FIG. 18). The use of projection 236 can have some benefits in that by adjusting the length and width of projection 236 that is received within guide channel 198, projection 236 can help restrict unwanted longitudinal and lateral movement of each carriage 220 on guide plate 182. As also shown in FIG. 15, rotatably mounted on top surface 224 of body 222 are a pair of spaced apart upper guide wheels 250A and 250B. For example, axles 252 project from top surface 224, such as orthogonal to axles 235 and parallel to axle 240, and upper guide wheels 250A and 250B can be rotatably mounted thereon. In one exemplary embodiment, each of travel wheels 232, 234, lower guide wheels 238, and upper guide wheels 250 each rotate on a bearing, such as a ball or needle bearing. The use of bearings decreases friction and enables the easy movement of carriages 220 under a low input force from motor 154 (shown in FIG. 10). As a result, the movement of carriages 220 and the dies 96 mounted thereon can be precisely controlled. In addition, the bearings can be sealed and designed to avoid the need for external lubrication, thereby making the assembly conducive for operation within a clean room.

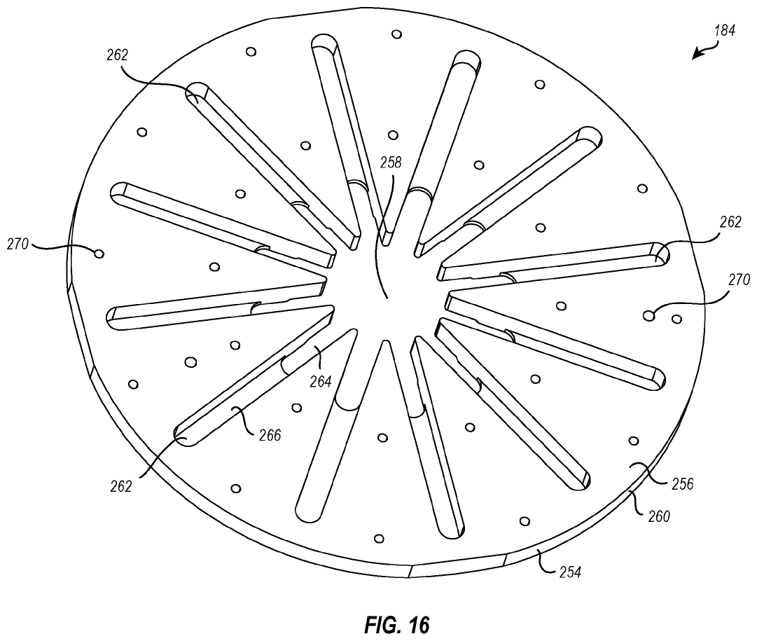

Turning to FIG. 16, cover plate 184 of expander plate assembly 158 has a top surface 254 and an opposing bottom surface 256 that extend between a central opening 258 and an outer perimeter edge 260. A plurality of upper guide channels 262 are formed on bottom surface 256 of cover plate 184 so to communicate with central opening 258. Upper guide channels 262 are elongated, linear and aligned with and radially outwardly project from central opening 258. Upper guide channels 262 are equally radially spaced apart around central opening 258 and the number of upper guide channels 262 is typically equal to the number of alignment channels 214/guide channels 198, discussed above. Upper guide channels 262 are configured to receive upper guide wheels 250 of corresponding carriages 220 (FIG. 15). In one embodiment, the full length of upper guide channels 262 can extend entirely through cover plate 184 between top surface 254 and bottom surface 256. However, in the depicted embodiment, only a first portion 264 of each upper guide channel 262 extending directly from central opening 258 passes entirely through cover plate 184. A second portion 266 of each upper guide channel 262 extending from first portion 264 is only recessed into bottom surface 256. This partial covering of upper guide channels 262 is optional but improves safety by coving moving parts and improving structural integrity of cover plate 184.

Bottom surface 256 of cover plate 184 sits on top of top surface 206 of channel plate 186 so that each upper guide channel 262 centrally aligns with a corresponding alignment channel 214. To help facilitate proper alignment and coupling between cover plate 184, channel plate 186, and guide plate 182, pins 268 can be formed projecting from top and/or bottom surface of cover plate 184, channel plate 186, and guide plate 182 while openings 270 configured to receive pins 268 can be formed on the opposing top and/or bottom surface of cover plate 184, channel plate 186, and guide plate 182 (shown in FIG. 13). Pins 268 and opening 270 can be positioned so that pins 268 can only be received within openings 270 when cover plate 184, channel plate 186, and guide plate 182 are properly aligned. Guide plate 182, channel plate 186 and cover plate 184 can be removably connected together by fasteners, such as screws, blots, clamps or the like or can be permanently connected together by welding, adhesive, press fit or the like. To withstand applied loads, guide plate 182, channel plate 186, cover plate 184, and spindle plate 162 are typically made of metal such as stainless steel or aluminum. However, other materials having the needed strength properties can also be used.

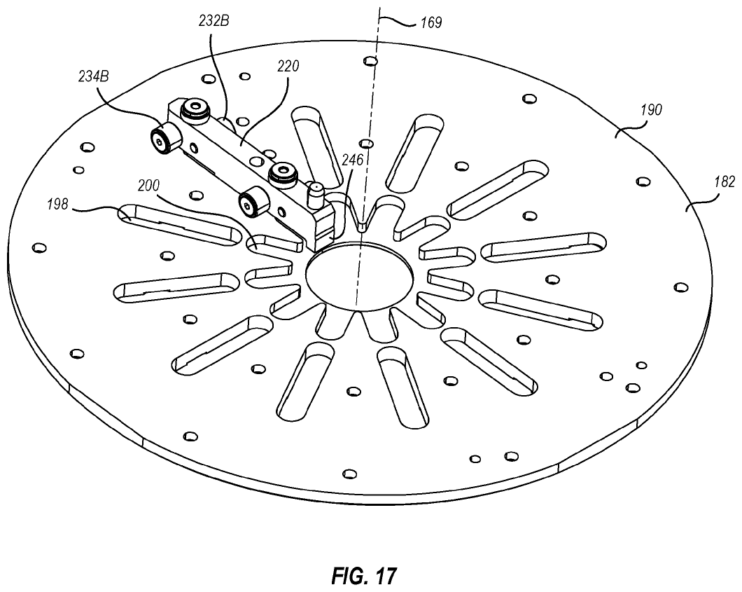

Although expansion mechanism 90 can be assembled using a variety of different sequential steps, in one method, spindle plate 162 is mounted to the end of drive shaft 164, such as through the use of flange 168, as previously discussed with regard to FIGS. 9 and 10. In this configuration, motor 154 can be used to selectively rotate spindle plate 162, relative to support 150, in opposite directions over a predefined angle. Next, carriages 220 are mounted on guide plate 182. Specifically, with reference to FIGS. 17 and 18, lower guide wheels 238 typically have a diameter larger than a width of guide channels 198 so that lower guide wheels 238 cannot pass through guide channels 198. Accordingly, with lower guide wheels 238 separated from body 222 of carriages 220, carriages 220 are positioned on top surface 190 of guide plate 182 so that nose 246 of each carriage 220 is received within a guide path 200 and projection 236 is received with the corresponding aligned guide channel 198. Lower guide wheels 239 are then secured to projections 236 within guide channels 198 from below bottom surface 192 through the use of axles 240. In this position with projections 236 having a length shorter than a length of guide channels 198, carriages 220 can freely roll linearly on top surface 190 of guide plate 182 between a contracted position toward axis 169 and an expanded position away from axis 169. Having noses 246 within guide paths 200 and projections 236 within guide channels 198 limits movement of carriages 220 along a linear path, i.e., restricts excessive lateral and longitudinal movement of carriages 220. Finally, the attachment of lower guide wheels 238 to projections 236 from bottom surface 192 prevents vertical separation of carriages 220 guide plate 182.

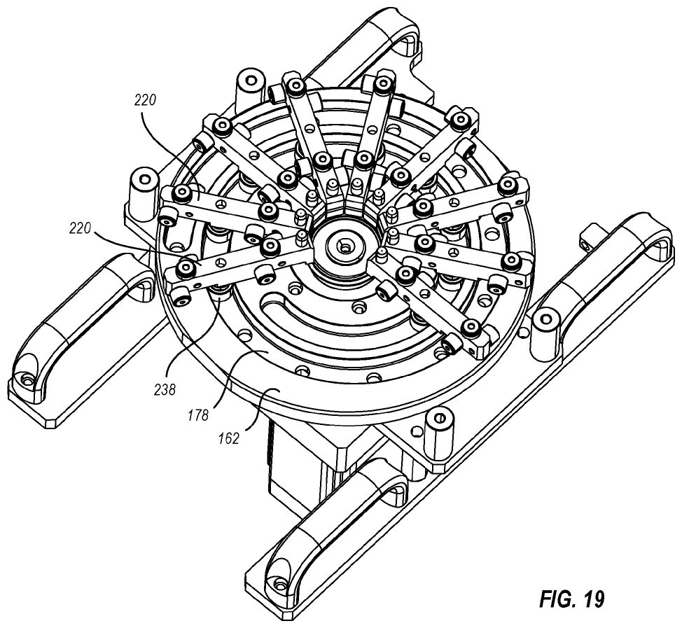

Returning to FIG. 14, channel plate 186 is positioned on top surface 190 of guide plate 182 so that each channel 214 partially encircles a corresponding carriage 220, i.e., channel plate 186 is positioned so that each carriage 220 is received within a corresponding channel 214. Channel plate 186 can be mounted on guide plate 182 before or after positioning carriages 220 on guide plate 182. Alternatively, as previously discussed, channel plate 186 can be integrally formed with guide plate 182 or integrally formed with cover plate 184. In still other embodiments, channel plate 186 can be eliminated as long as a gap of formed between guide plate 182 and cover plate 184 to receive carriages 220. Channels 214 are large enough to enable each carriage 220 to move between its desired expanded and contracted positions. However, the walls of channel plate 186 bounding each channel 214 provide further protection against excessive radial/longitudinal displacement or lateral displacement of each carriage 220. Prior to or after positioning of channel plate 186 on guide plate 182, guide plate 182 is secured to support 150, such as by using braces 160 so that guide plate 182 is disposed directly above spindle plate 162. More specifically, as shown in FIG. 19 where guide plate 182, channel plate 186 and select carriages 220 have been hidden to improve clarity, guide plate 182 with carriages 220 are secured above spindle plate 162 so that each of lower guide wheels 238 are received within expansion channel 178 of spindle plate 162. The diameter of each lower guide wheel 238 is slightly smaller than the width of expansion channel 178 so that lower guide wheels 238 can freely roll against one of the opposing side surfaces bounding expansion channel 178.

Finally, as shown in FIG. 10, cover plate 184 is secured on top of channel plate 186, as previously discussed, so that upper guide wheels 250 of each carriage 220 are received within a corresponding upper guide channel 262 and so that mounting pins 248 of each carriage 220 are received within or are aligned with exposed first portion 264 of each upper guide channel 262. Again, the upper guide wheels 250 are slightly smaller than the width of upper guide channels 262 so that guide wheels 250 can freely rotate against one of the opposing sidewalls of upper guide channels 262. The capturing of upper guide wheels 250 within upper guide channels 262 helps to further limit movement of carriages 220 along the linear path between the contracted and extended positions.

With reference to FIGS. 10, 14 and 19, in the above assembled configuration, motor 154 can be activated to rotate spindle plate 162 in a first direction over a predefined angle. As spindle plate 162 is rotated, lower guide wheels 238 travel along expansion channel 178 toward the radially enlarged end of expansion channel 178. Concurrently, as lower guide wheels 238 are traveling along expansion channel 178, because of the radial expansion of expansion channel 178, guide wheels 238 are also being moved radially outward away from axis 169 which, in turn, moves all of carriages 220 radially outward to an expanded position. In contrast, motor 154 can then be activated to rotate spindle plate 162 in a second direction opposite to the first direction over a predefined angle. Again, as spindle plate 162 is rotated by motor 154, lower guide wheels 238 travel along expansion channel 178 toward the radially constricted end of expansion channel 178. Concurrently, as guide wheels 238 are traveling along expansion channel 178, guide wheels 238 are also being moved radially inward toward axis 169, which in turn moves all of carriages 220 radially inward to the contracted state. Mounting pins 248 on carriages 220 move along exposed first portion 264 of each upper guide channel 262 as carriages 220 are moved between the expanded and contracted positions. The extent of movement of carriages 220 radially inward and radially outward is dependent upon the angle or rotation imparted to drive shaft 164 and spindle plate 162 by motor 154. That is, dependent upon the size of compression collar 60A and the amount of desired expansion thereof, motor 154 can be used to impart different lengths of radial displacement of carriages 220.

Although expansion channel 178 on spindle plate 162 is shown in FIG. 11 as a single spiral channel, other configurations can also be used. For example, in other embodiments expansion channel 178 can comprise two or more separate concentric channels on top surface 170 that encircle or at least partially encircle opening 174 with each channel having a spiral configuration. One or more of the guide wheels 238 can be received within each of the two or more channels. In still other embodiments, depending on the needed angle of rotation for spindle plate 162, expansion channel 178 can comprise a plurality of separate arced or curved channel sections the each only partially encircle opening 174. A separate guide wheel 238 (shown in FIG. 19) can be positioned within each of the plurality of separate arced or curved channel sections. It is also noted that the guide wheels 238 can be positioned at different locations along elongated body 222 so that the plurality of separate arced or curved channel sections can be placed at different radial distances from opening 174. Other configurations can also be used.

It is understood that the use of spindle plate 162 with a single motor 154 to facilitate the radial displacement of carriages 220 has a number of unique benefits. For example, spindle plate 162 is a simple element having minimal parts that efficiently and concurrently radially displaces a plurality of carriages. The use of spindle plate 162 also provides a mechanical advantage and enables easy movement of carriages 220 in opposite directions. The use of spindle plate 162 with motor 154 also makes it easy to control the distance and rate of movement of carriages 220 and to hold carriages 220 in any expanded position for a desired period of time. Other benefits also exist. However, in alternative embodiments, it is appreciated that spindle plate 162 and motor 154 can be replaced with other mechanisms for radially displacing carriages 220. For example, individual drive systems can be mounted to each carriage 220 for radially displacing each carriage. Such drive systems can include a gear and chain system, pully system, screw system, hydraulic or pneumatic rail system, a movably track system or the like.

With expansion mechanism 90 assembled and in operation within housing 12, die set 92 can be removably coupled thereto. Although not required, attachment of die set 92 is typically simpler if expansion mechanism 90 is positioned so that carriages 220 are in their contracted or home state. An operator can then manually pass die set 92 down through access opening 30 and couple it with expansion mechanism 90. Specifically, with reference to FIGS. 5, 6 and 10, the central openings of guide plate 182, channel plate 186, and cover plate 184 are vertically aligned to form a central recess 274 on expander plate assembly 158 in which die set 92 can be received. Mounting pins 248 upwardly projecting on carriages 220 are received with central recess 274 or within the exposed first portion 264 of corresponding upper guide channels 262 of cover plate 184. Each upper guide channel 262 extends to and communicates with central recess 274. During the mounting process, die set 92 is orientated vertically over central recess 274 so that each die 96 is aligned with a corresponding carriage 220. Die set 92 is then lowered into central recess 274 until each die 96 rests on top of body 222 of a corresponding carriage 220 and the mounting hole 110 of each die 96 receives mounting pin 248 of the corresponding carriage 220. Leg 130 of each die 96 is received within central recess 274.

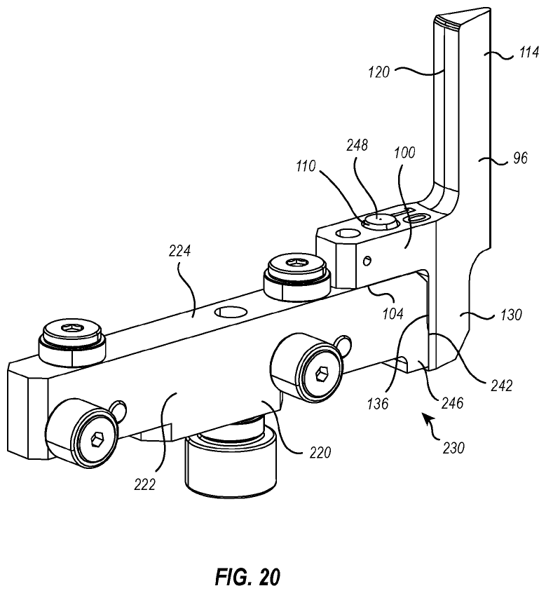

More specifically, as depicted in FIG. 20, each die 96 is mounted on a corresponding carriage 220 so that bottom surface 104 of base 100 rests on top of top surface 224 of body 222 at first end 230, outside face 136 of leg 130 of die 96 rests against or is disposed directly adjacent to first end face 242 of body 222, and mounting pin 248 of each carriage 220 is received within a corresponding mounting hole 110 of each die 96. In this configuration, each die 96 is securely and stably mounted on a corresponding carriage 220 and any rotation between die 96 and carriage 220 is precluded or at least limited. For example, the positioning of flat outside face 136 of die 96 against flat first end face 242 of body 222, precludes or at least limits any rotation between each die 96 and the corresponding carriage 220. One of the benefits of the disclosed configuration of die set 92 and expansion mechanism 90 is that it permits simultaneous mounting of all dies 96 onto the carriages 220 and the simultaneous removal of all dies 96 from the carriages 220 without the need for attaching or removing separate fasteners. Furthermore, each die 96 can be mounted to any carriage 220 so no specific orientation of die sets 92 is needed.

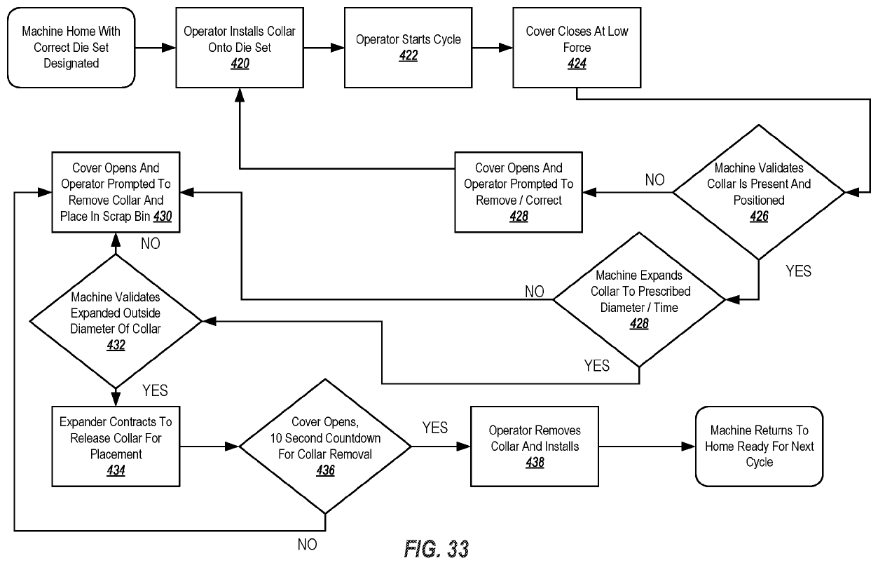

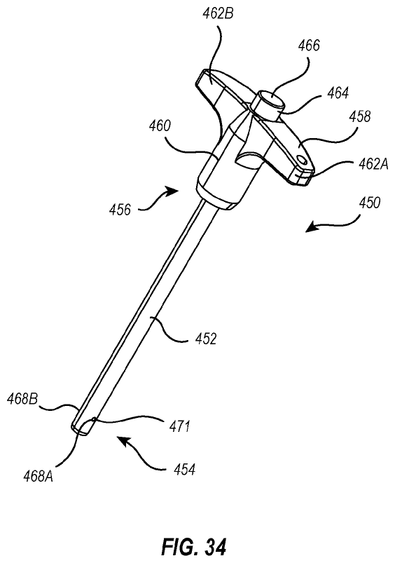

As referenced above, die set 92 and/or the individual dies 96 can be manually grasped by an operator and placed on expansion mechanism 90/carriage 220. In alternative embodiments, an operator can use a placement tool to individually grasp dies 96 and place them on or remove them from expansion mechanism 90/carriage 220. For example, depicted in FIG. 34 is one exemplary embodiment of a placement tool 450 that can be used for grasping and placing dies 96. Placement tool 450 includes a tubular stem 452 having a distal end 454 and an opposing proximal end 456. Mounted on proximal end 456 is a handle 458. In the depicted embodiment, handle 458 includes a central body 460 and a pair of finger rests 462A and 462B radially outwardly projecting from opposing sides thereof.

A spring biased plunger 464 is slidably disposed within stem 452 and has a terminal end 466 the projects proximal of handle 458. Small openings 468A and 468B extend through the side of stem 452 at distal end 454. A pair of balls 469 are disposed within stem 452 and are each aligned with a corresponding one of openings 468A and 468B. During operation, an operator distally presses plunger 464 into stem 452 so as to be in a depressed position. When in the depressed position, balls 469 can freely move radially inward so that they do not project out of openings 468A and 468B. When plunger 464 is released, a spring resiliently urges plunger 464 back to a retracted position. When in the retracted position, plunger 464 presses balls 469 radially outward so that they partially project out through corresponding openings 468A and 468B and past the exterior surface of stem 452.

With reference to FIGS. 7B and 34, each die 96 includes holes 470A and 470B that extend laterally through opposing sides of base 100 so as to extend to and communicate with placement hole 112. Distal end 454 of stem 452 of placement tool 450 is sized to be slidably received within placement hole 112 of dies 96. More specifically, during use an operator moves plunger 464 into the depressed position and advances distal end 454 of stem 452 into placement hole 112 of a corresponding die 96 so the openings 468A and B are aligned with holes 470A and B. Plunger 464 is then released which moves plunger 464 back to retracted position and forces balls 469 to project out through opening 468 and into holes 470, thereby securing die 96 to placement tool 450. The operator can then use placement tool 450 to easily and properly position die 96 on a carriage 220 (shown in FIG. 20). Once die 96 is positioned, the operator again moves plunger 464 back to the depressed position which enables placement tool 450 to be freely removed from placement hole 112 and separated from die 96. The same process can also be used to remove dies 96 from expansion mechanism 90/carriage 220. Other placement tool configurations can also be used.

With die set 92 coupled to carriages 220, dies 96 of die set 92 concurrently move radially with carriages. More specifically, as carriages 220 move radially between their expanded and contracted positions by rotation of spindle plate 162, dies 96 of die set 92 also concurrently move radially between their expanded and contracted positions.

Turning to FIG. 23 is an enlarged view of compression collar 60A that can be selectively expanded by expander assembly 10/expansion mechanism 90, as discussed above. Compression collar 60A includes a tubular body 300 extending between a first end 302 and an opposing second end 304. First end 302 terminates at a first end face 306 while second end terminates at a second end face 308. Tubular body 300 has interior surface 310 and an opposing exterior surface 312 that extend between opposing ends 302 and 304. Interior surface 310 bounds a throughway 314 extending through body 300 and, more specifically, extends between opposing end faces 306 and 308. Throughway 314 typically has a circular transverse cross section at all points between opposing ends 302 and 304. Throughway 314 also typically has a constant diameter at all points between opposing ends 302 and 304. In some embodiments, the diameter may taper slightly extending to one of ends 302 or 304 to better facilitates some methods of production, such as injection molding. Throughway 314 will typically have a constant diameter over at least or less than 40%, 60%, 80%, 90%, 95%, or 98% of the length of throughway 314 or in a range between any two of the foregoing.

Compression collar 60A can be formed having a variety of different sizes depending on intended use and depending on the size of the tube to be used with compression collar 60A. In some embodiments, the maximum diameter of throughway 314 can be at least or less than 2 mm, 3 mm, 4 mm, 6 mm, 8 mm, 10 mm, 15 mm, 20 mm, 25 mm, 30 mm, 40 mm, 50 mm, 60 mm, 70 mm, or 90 mm or in a range between any two of the foregoing. Other dimensions can also be used. Compression collar 60A can also have a length extending between end faces 306 and 308 that can be at least or less than 2 mm, 3 mm, 4 mm, 6 mm, 8 mm, 10 mm, 15 mm, 20 mm, 25 mm, 30 mm, 40 mm, 50 mm, 60 mm, 70 mm, or 90 mm or in a range between any two of the foregoing. Other dimensions can also be used. Commonly, although not required, the length of compression collar 60A is typically greater than the diameter.

Compression collar 60A further includes a first spacer tab 318A and a spaced apart second spacer tab 318B outwardly projecting from first end 18 of tubular body 12 and, more specifically, outwardly projecting from first end face 306. In one exemplary embodiment, spacer tabs 318A and 318B are opposingly facing and are disposed on opposing sides of first end face 306. Each spacer tab 318A and 318B terminates at a terminal end face 320 and has shoulders 322 and 324 that extend from opposing ends of terminal end face 320 to first end face 306. Compression collar 60A includes a first window 326A bounded between shoulders 322 of spacer tabs 318A and 318B and a second window 326B bounded between shoulders 324 of spacer tabs 318A and 318B. Windows 326A and 326B laterally pass-through compression collar 60A so as to communicate with throughway 314.

It is appreciated that spacer tabs 318 can have a variety of different widths, i.e., the distance between shoulders 322 and 324 that spacer tab 318 extends along first end face 306. For example, with reference to the full annular length of first end face 306, each spacer tab 318 can have a width that is at least or less than 5%, 10%, 15%, 20%, 30%, 40%, 50%, 60% or 70% of the full annular length of first end face 306 or is in a range between any two of the percent values. Spacer tabs 318 also typically have a height H2 extending between first end face 306 and terminal end face 320 that is at least or less than 0.5 mm, 1 mm, 1.5 mm, 2 mm, 3 mm, 4 mm, 5 mm, 7 mm, 10 mm, 15 mm, 20 mm or is in a range between any of the two foregoing values. Height H2 can vary based on the diameter of tubular body 300. For example, in some embodiments the height H2 can increase as the diameter increases.

Compression collar 60A is typically comprised of a polymeric material having memory properties, i.e., the material will resiliently rebound towards its original shape when stretched. One common example of a polymeric material having memory properties that can be used to form compression collar 60A is cross-linked polyethylene that is commonly abbreviated as PEX. PEX is commonly formed from high-density polyethylene (HDPE). PEX contains cross-linked bonds in the polymer structure that change the thermoplastic to a thermoset. Depending on the manufacturing process and the specific type of material used to form compression collar 60A, the cross-linking can be accomplished prior to, during or after the forming of compression collar 60A. The required degree of cross-linking is typically between 65% and 89%. A higher degree of cross-linking could result in brittleness and stress cracking of the material, while a lower degree of cross-linking could result in product with poor physical properties.

For some cross-linking materials, e.g., some HDPE materials, the cross-linking or at least a majority of the cross-linking can automatically be achieved during the manufacture process, especially where the material forming the compression collar is heated during the forming process. A Silane or “moisture cure” method can also be used to further facilitate the desire cross-linking. In this method, the formed compression collars are placed in a heated water bath or in a heated environmental chamber having a relative humidity of between 60% and 98% and allowed to cure for a sufficient time to achieve the desired cross-linking. Other applications of heat and moisture can also facilitate the needed cross-linking.

For some alternative cross-linking materials, the cross-linking can be accomplished by applying radiation, such as electron beam radiation (ebeam), to the polymer, as is commonly known in the art. For example, in one method of cross-linking the polymer, compression collar 10 is subject to at least or less than 50 kGy, 60 kGy, 70 kGy or 80 kGy of ebeam or in a range between any two of the foregoing, after being molded. Other amounts can also be used. A mechanical ebeam generator can be used to generate the ebeam.

In one method of manufacture, compression collar 60A can be formed by a molding process such as injection molding. The injection molding process heats the material which can facilitate at least a majority of the needed cross-linking. Using an injection molding process enables the compression collar 60A to be easily formed with rounded corners so as to avoid or limit sharps. Typically, compression collar 60A will be molded and then subjected to post cross-linking process, such as discussed above. However, the desired cross-linking can be achieved during the initial manufacturing process either as a result of the manufacture process and/or by applying heat and/or humidity during manufacture and/or applying radiation during manufacture. It is appreciated that other molding processes such as blow molding, rotational molding, and the like can also be used to form compression collar 60A. Other manufacturing processes can also be used to form compression collar 60A. For example, compression collar 60A could be machined or cut from an extruded tube of material. Other methods can also be used.

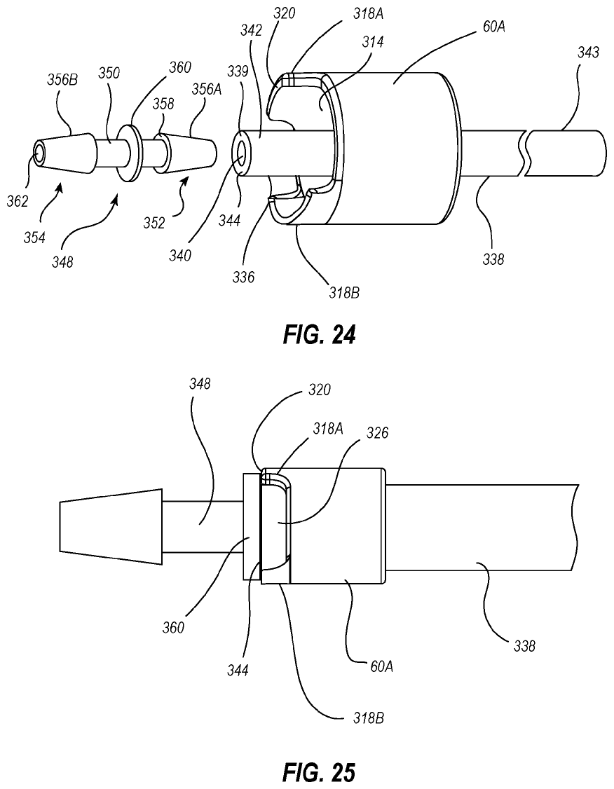

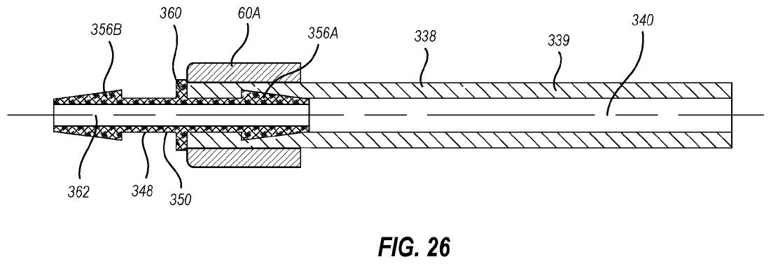

As depicted in FIGS. 24-26, compression collar 60A can be used to secure a tube 338 to a tube fitting 348 so that a liquid tight seal is formed between tube 338 and tube fitting 348. More specifically, tube 338 comprises an encircling side wall 339 having an interior surface and an opposing exterior surface. The interior surface bounds a passage 340 extending along the length of tube 338. Tube 338 has a first end 342 that terminates at a terminal end face 344. Tube 338 is also typically made of a polymeric material having memory properties. Although in some embodiments tube 338 can be made of the same material as compression collar 60A, as discussed above, tube 338 is commonly made from a material that is different from the material for compression collar 60A. Typically, the material for tube 338 has a modulus of elasticity that is lower than the modulus of elasticity for the material of compression collar 60A. That is, the material for tube 338 is typically more flexible than the material used to form compression collar 60A. Examples of materials that can be used for tube 338 that have a lower modulus of elasticity include silicone, polyvinyl chloride (PVC), and thermoplastic elastomers (TPE). Other materials can also be used. It is appreciated that tube 338 can have any desired diameter and any desired length.

The term “tube fitting” as used in the specification and appended claims is broadly intended to include any type of fitting or other structure designed for coupling with tube 338. For example, tube fitting 348 could comprise a coupling fitting, union fitting, port fitting, plug fitting, T-fitting, Y-fitting, elbow fitting, reducer fitting, adapter fitting or the like. Tube fitting 348 may be a standalone structure or may be attached to or be configured to be attach to another structure such as a bag, container, tube, or other fitting. Commonly, at least a portion of tube fitting 348 is designed to be received within passage 340 of tube 338 for making a connection therewith. It is also common that tube fitting 348 is tubular so that a sealed fluid connection can be formed between tube fitting 348 and tube 338. In other embodiments, however, such as where tube fitting 348 is a plug, tube fitting 348 need not be tubular.

In the depicted embodiment, tube fitting 348 comprises a coupling fitting used to fluid couple two separate tubes together. Tube fitting 348 comprises a stem 350 having a first end 352 and an opposing second end 354. Formed on and radially encircling an exterior surface of stem 350 at first end 352 is an annular barb 356A having a frustoconical configuration. Barb 356A includes an annular outside shoulder 358. Although stem 350 is shown having a single barb 356A formed thereon, in other embodiment, stem 350 can be formed with at least or less than one, two, three, four or more consecutive or spaced apart barbs 356A formed thereon. Formed on and radially encircling the exterior surface of stem 350 at second end 354 is an annular barb 356B having the same configuration and elements as barb 356A. Again, stem 350 can be formed with at least or less than one, two, three, four or more barbs 356B formed thereon. Although not required, a flange 360 having opposing side faces encircles and radially outwardly extends from stem 350 at a location between barbs 356A and 356B. As also shown, stem 350 can be tubular having an interior surface that bounds a passage 362 that extends through stem 350 between opposing ends 352 and 354.

Tube fitting 348 is typically molded from a polymeric material. However, other materials and molding processes can also be used. Tube fitting 348 is also typically made from a material that is different from the material used to form tube 338 and compression collar 60A. In addition, the material used to form tube fitting 348 typically has a modulus of elasticity that is greater than the modulus of elasticity of the materials used to form tube 338 and compression collar 60A. That is, tube fitting 348 is typically less flexible than tube 338 and compression collar 60A.

In one method of use, compression collar 60A is initially radially expanded using expander assembly 10/expansion mechanism 90. Turning to FIG. 24, once compression collar 60A is in the expanded state, first end 342 of tube 338 bounding passage 340 is advanced into throughway 314 of compression collar 60A.

Before or after first end 342 of tube 338 is advanced into throughway 314 of compression collar 60A, first end 352 of tube fitting 348 is advanced into a passage 340 of tube 338. Tube fitting 348 is typically advanced into passage 340 prior to significant constricting of compression collar 60A so that compression collar 60A does not interfere with the insertion of tube fitting 348. Furthermore, tube 338 is typically sufficiently flexible that tube fitting 348 can be manually pressed into passage 340. In other embodiments, however, a tool or machine can be used to assist in the insertion of tube fitting 348.

Tube fitting 348 is typically advanced until a side face of flange 360 butts against the terminal end face 344 of tube 338. As needed, the assembled tube fitting 348 and tube 338 are moved so that terminal end faces 320 of spacer tabs 318A and 318B butt against the side face of flange 360 of tube fitting 348. That is, tube fitting 348 and tube 338 can be assembled outside of compression collar 60A and then moved into place. Alternatively, tube 338 or tube fitting 348 can be held at the desired location relative to compression collar 60A while the other of tube 338 or tube fitting 348 is coupled thereto. In this method, no movement of tube 338 or tube fitting 348 is required relative to compression collar 60A once tube 338 and tube fitting 348 are coupled together. After tube fitting 348 and tube 338 are properly positioned, compression collar 60A is left to automatically, resiliently rebound back toward the contracted state. At a minimum, compression collar 60A resiliently rebounds so as to have an inner diameter that is smaller than the outer diameter of tube 338, thereby compressing tube 338. As compression collar 60A resiliently constricts, it radially inwardly pushes and constricts tube 338 so as to form a uniform, annular, liquid tight seal between tube 338 and barb 356A.

Windows 326 and 328 enables a visual inspection of first end 342 of tube 338 to ensure that first end 342 of tube 338 remains adjacent to or butted against flange 360 while compression collar 60A is positioned adjacent to or butted against flange 360, thereby ensuring that both tube 338 and tube fitting 348 are properly positioned within compression collar 60A so that compression collar 60A produces the desired liquid tight seal between tube 338 and tube fitting 348.

Depending on the situation, variations in the assembly process may also be used. For example, if tube 338 has a free second end 343 that is opposite first end 342, tube 338 and tube fitting 348 could be coupled together outside of compression collar 60A. Once assembled, second end 343 could be advanced through throughway 314 of compression collar 60A until spacer tab 318A and 318B butt against flange 360 of tube fitting 348. In yet another alternative, compression collar 60A may be configured so that in the expanded state, tube fitting 348 (including flange 360) can pass entirely through throughway 314. In this embodiment, tube 338 and tube fitting 348 could again be coupled together outside of compression collar 60A. Once assembled, tube fitting 348 having tube 338 therein could be advanced through compression collar 60A until the side face of flange 360 is aligned with terminal end face 320 of spacer tabs 318A and 318B. The assembly could then be held in this position until compression collar 60A sufficiently constricts so that spacer tab 318A and 318B butt against flange 360. Other methods of assembly can also be used depending on the facts.

In an alternative embodiment, it is appreciated that compression collar 60A can be formed with 1, 3, 4, or more spaced apart spacer tabs 318 or spacer tabs 318A and 318B can be eliminated. For example, depicted in FIG. 27 is a compression collar 60B. Compression collar 60B has the same structural elements and is used in the same way as compression collar 60A except that compression collar 60B eliminates second spacer tab 318B so as to only include first spacer tab 318A projecting from first end 302 of tubular body 300. Spacer tab 318A bounds opposing ends of a single window 326A that extends between opposing shoulders 322 and 324 of first spacer tab 318A.

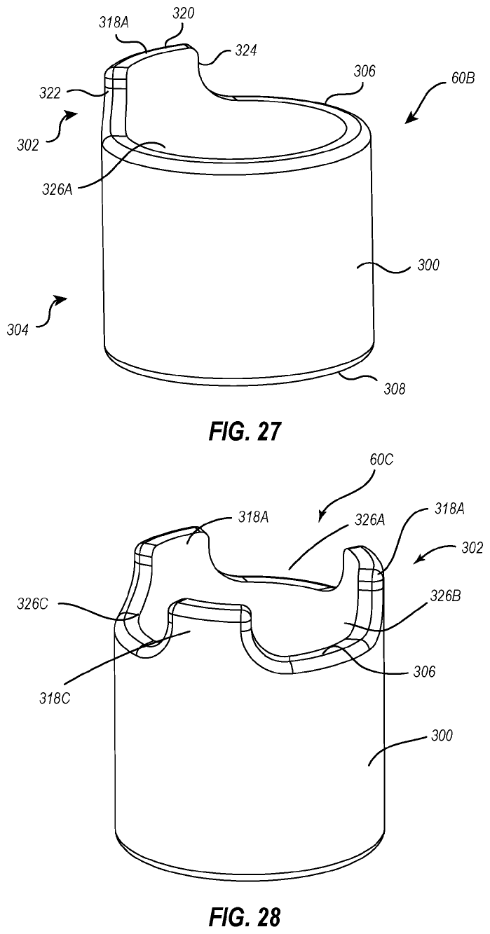

In another alternative embodiment depicted in FIG. 28, a compression collar 60C is provided. Compression collar 60C has the same structural elements and is used in the same way as compression collars 60A and 60B except that compression collar 60C also includes a third spacer tab 318C longitudinally projecting from first end face 306 of tubular body 300. Again, spacer tabs 318A, 318B, and 318C are shown projecting from first end face 306 and bound windows 326A, 326B, and 326C therebetween.

Further alternative embodiments of compression collars and related methods of use that can be used with expander assembly 10/expansion mechanism 90 are disclosed in US Patent Publication No. 2018/0187809, published Jul. 5, 2018, which is incorporated herein by specific reference.

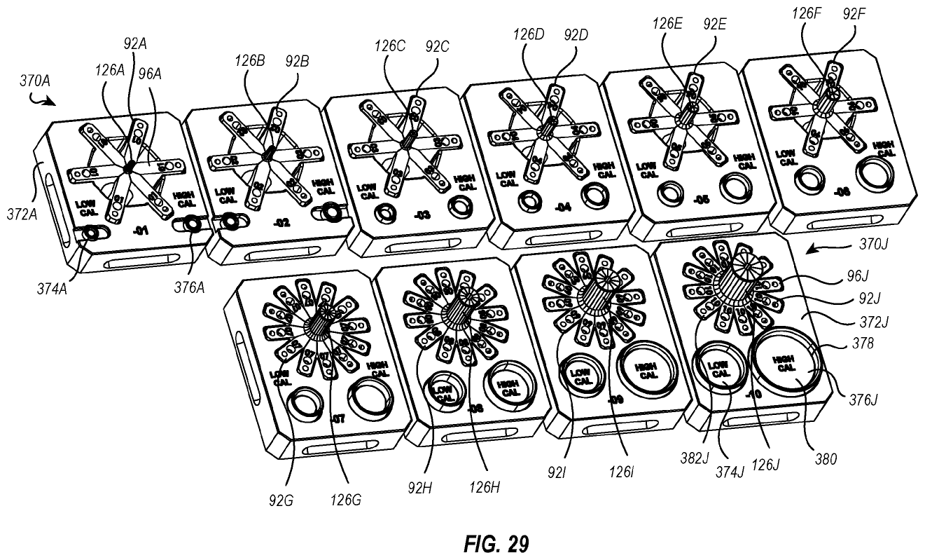

Depicted in FIG. 29 are a plurality of die sets 92A-92J which can each be used as a replacement to previously discussed die set 92 and which each have the same structural components as die set 92 and can be used in the same way as die set 92. As such, like elements between die set 92 and each of die sets 92A-92J are identified by like reference characters. Specifically, each die set 92A-92J comprises a plurality of dies 96 that can couple to and operate on expander assembly 10/expansion mechanism 90, as previously discussed with regard to die set 92, for expanding compression collar 60A or any other compression collars discussed herein. In general, die sets 92A-92J differ from each other in that they each have a different size die head. Specifically, die sets 92A-92J have die heads 126A-126J, respectively. Die heads 126A-126J differ from each other in that they each have a different diameter D1 and/or height H1 (see FIG. 6). Specifically, in the depicted embodiment, the diameter D1 and height H1 of die heads 126A-126J progressively enlarge for each consecutive die set 92A-92J. The potential sizes and ranges of diameter D1 and height H1 for each die head 126A-126J is the same as that previously discussed with regard to die head 126. Some of die sets 92A-92J have more dies 96 and corresponding prongs 114 than others. For example, die sets 92A-92F are depicted as each having six dies 96 while die sets 92G-92J each have twelve dies 96. In other embodiments, each of die sets 92A-92J can have other numbers of dies 96, such as previously discussed with regard to die set 92. However, other than the size of prongs 114/die heads 126, the remainder of each die 96 for each die set 92A-92J has the same or substantially the same configuration as previously discussed with die 96 so that they can also be used on the same expander assembly 10/expansion mechanism 90. That is, all of die heads 126A-126J engage with carriages 202.

Each die set 92A-92J/die head 126A-126J is designed to operate with a different size or differ range of sizes of compression collars 60. As previously discussed, compression collars 60 can come in a plurality of different sizes depending on their intended use. In view of the potential large range of different sizes of compression collars 60, a plurality of different sizes of die sets can be used so that each die set optimally expands one specific size of compression collar 60A or a narrow range of different sizes of compression collars 60. For example, each die sets 92A-92J may be configured to expand at least 1, 2, 3, 4, 5, 6, 7, 8 or more different sizes of compression collars 60 or in range between any two of the foregoing. In one embodiment, each of die sets 92A-92J may be configured to expand between 3 and 7 or more commonly between 4 and 6 different sizes of compression collars 60.

In one embodiment of the present disclosure, a compression collar expander system is provided. The system includes expander assembly 10 and a plurality of different sizes of die sets 92. That is, expander assembly 10 and the plurality of different sizes of die sets 92 may be sold together as a type of kit forming a system. In the example illustrated in FIG. 29, the system can be provided with die sets 92A-92J, as discussed above. In other embodiments, depending on the intended use, the system can be provided with more or fewer die sets of different sizes. For example, in alternative embodiments, a compression collar expander system can include expander assembly 10 and at least 2, 4, 6, 8, 10, 12, or 14 die sets 92 of different sizes or in a range between any two of the foregoing.

As also depicted in FIG. 29, each die set 92A-92J can be provided as part of a tray assembly. For example, a tray assembly 370J includes a tray 372J having die set 92J, a low calibration ring 374J, and a high calibration ring 376J, each removably mounted on tray 372J. Tray 372J can be provided with complementary slots and/or projections for securely receiving die set 92J, low calibration ring 374J and high calibration ring 376J. Each calibration ring 374J and 376J comprises an annular ring which, as discussed below, can be used for verifying that expander assembly 10 is properly calibrated for use with die set 92J. Calibration rings 374J and 376J are typically made of metal, such as aluminum or stainless steel, although other rigid materials, such as rigid plastics or composites, can also be used. Each calibration ring 374J and 376J has an annular interior surface 378 that encircles a passage 380 extending therethrough and having a diameter. Interior surface 378 and passage 380 are typically circular with the diameter of passage 380 of high calibration ring 376J being greater than the diameter of low calibration ring 374J. More specifically, in one exemplary embodiment, the diameter of interior surface 378 of low calibration ring 374J can correspond to the inner diameter of the smallest compression collar 60 designed for use on die set 92J when the smallest compression collar 60 has been expanded by die set 92J while the diameter of interior surface 378 of high calibration ring 376J can correspond to the inner diameter of the largest compression collar 60 designed for use on die set 92J when the largest compression collar 60 has been expanded by die set 92J.

As depicted, each of the other die sets 92A-92K can also be formed as part of a tray assembly with a corresponding tray, low calibration ring and high calibration ring. Like numbers can be used to identify like elements between the different tray assemblies. As each die set 92A-92J has a different sized die head 126, the calibration rings are different sizes for each tray assembly 370A-370J.

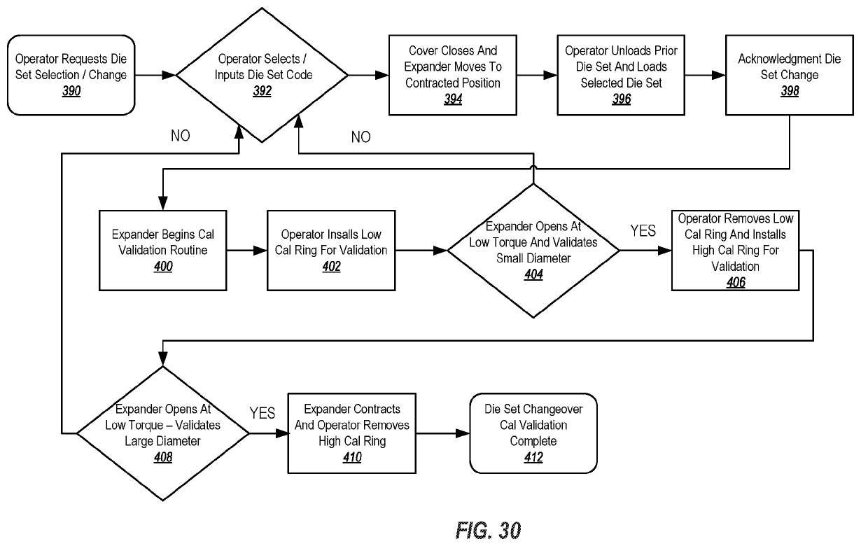

FIG. 30 is a flow chart providing one example of how to validate the calibration of expander assembly 10, shown in FIG. 1, when using a select one of die set 92A-92J, shown in FIG. 29. In step 390, an operator activates expander assembly 10 and requests die set selection/change. All inputs by the operator can be through touch input on display screen 44 and/or through control switches 46. In step 392, the operator is prompted for and either selects or inputs into expander assembly 10 a code corresponding to a select die set 92 chosen from die sets 91A-92J. The selected die set is chosen based on the desired size of compression collar 60 to be used. In the present example, die set 92J is the selected die set. The code can comprise an alpha and/or a numeric code with is typically labeled directly on each die set. See, for example, code 382J corresponding to “10” on die set 92J in FIG. 29. In step 394, following the selection/input of the die set code, expander assembly 10 is programmed to automatically move cover 54 to the closed position (see FIG. 1) and, if not already done, move expander mechanism 90 to the contracted position, as shown in FIG. 6. In step 396, cover 54 is then automatically moved to the open position (see FIG. 2) and the operator manually reaches down through access opening 30 and removes any die set currently mounted on expansion mechanism 90. The operator then grasps selected die set 92J, manually passes it down through access opening 30, and mounts it on expansion mechanism 90, as previously discussed with regard to FIG. 20 and shown in FIG. 6. As previously discussed, die sets 92 can be moved by using placement tool 450 (see FIG. 34) to sequentially move each of dies 96. In step 398, one or more sensors within housing 12 of expander assembly 10 can be used to automatically detect that the selected die set 92J has been properly placed on expansion mechanism 90. For example, as shown in FIG. 5, optical sensors 386A and/or 386B, such as laser micrometers, can be used to detect the presence of the newly mounted die set 92J and confirm that it has been properly mounted. The placement of the selected die set 92J can be acknowledged on display screen 44. In one exemplary embodiment, optical sensors 386A and/or 386B can scan using a light beam that extends either laterally across die set 92J or vertically along die set 92J.

In step 400, expander assembly 10 now begins a calibration validation routine that is specific for selected die set 92J. In step 402, the operator is prompted to and facilitates mounting of low calibration ring 374J on die head 126J of die set 92J. More specifically, with cover 54 in the open position, the operator manually passes low calibration ring 374J down access opening 30 and slides low calibration ring 374 over die head 126J so that low calibration ring 374J freely rest at or towards the bottom of die head 126J.

In step 404, the operator can input that the low calibration ring 374J has been installed and/or sensors 386A and/or 386B can detect mounting thereof. Cover 54 then moves to the closed position and expansion mechanism 90 is automatically operated to outwardly expand die set 92J a predetermined distance. Specifically, expander assembly 10 using motor 154 automatically radially expands die set 92J a predetermined distance that is equal to the distance that die set 92J would expand when expanding a first diameter compression collar 60 designed to operate with die set 92J. The first diameter compression collar 60 is typically the smallest diameter compression collar designed to operate with die set 92J. Expander assembly 10 then measures, directly or indirectly, the force being applied by die set 92J against the interior surface of low calibration ring 374J. In one embodiment, the applied force can be measured by using a servo motor as motor 154. Expander assembly 10 then compares the measured force to a known predetermined force that die set 92J would actually be applying to the first diameter compression collar 60 when expanding the first diameter compression collar 60 by the same distance. If the compared forces are within a predefined tolerance, expander assembly 10 is properly calibrated for expanding the first diameter compression collar 60 and the process moves to step 406. If not, expander assembly 10 is not properly calibrated for expanding the first diameter compression collar 60. If the validation process fails, the process can return to step 392 to repeat the validation process or further recalibration steps can be taken.

In step 406, cover 54 is moved to the opened position. The operator then manually removes low calibration ring 374J and replaces it with high calibration ring 376J. Again, the placement of high calibration ring 376J can be automatically detected by sensors 386 and/or notice inputted by the operator. Cover 54 is then moved to the closed position. In step 408, the same process discussed above with regard to low calibration ring 374J in step 404 is now repeated for high calibration ring 376J. Specifically, expander assembly 10 automatically radially expands die set 92J a predetermined distance that is equal to the distance that die set 92J would expand when expanding a second diameter compression collar 60 designed to operate with die set 92J. The second diameter compression collar 60 has a larger inner diameter than the first diameter compression collar and is typically the largest diameter compression collar designed to operate with die set 92J. Expander assembly 10 then measures, directly or indirectly, the force being applied by die set 92J against the interior surface of high calibration ring 374j. Again, the applied force can be measured by using a servo motor as motor 154. Expander assembly 10 then compares the measured force to a known predetermined force that die set 92J would actually be applying to the second diameter compression collar 60 when expanding the second diameter compression collar 60 by the same distance. If the compared forces are within a predefined tolerance, expander assembly 10 is properly calibrated for expanding the second diameter compression collar 60. If not, expander assembly 10 is not properly calibrated for expanding the second diameter compression collar 60. In the latter case, the process can return to step 392 to repeat the validation process or further recalibration steps can be taken.

If the validation process using the high calibration ring 376J is successful, the process moves to step 410 where die set 92J is moved to the contracted position and the high calibration ring 376J is removed. In step 412 the die set change over calibration is complete. Once the validation process is completed and satisfied using low calibration ring 374J and high calibration ring 376J, calibration for expander assembly 10 using die set 92J is also verified through extrapolation for expanding other predetermined compression collars 60 having an inner diameter with a size that is between the size of the inner diameter of the first diameter compression collar 60 and the size of the inner diameter of the second diameter compression collar 60. Such other predetermined compression collars 60 can be at least 1, 2, 3, 4, or more other compression collars having an inner diameter with a size between the first compression collar and the second compression collar. As a result, the validation process is not required for all compression collars being expanded by the same die set. If it is desired to expand a compression collar 60 that is designed to operate with a different sized die set, the above process steps 390-412 are repeated for the new die set.

In an alternative exemplary embodiment, a single calibration ring can be used with each die set 92A-92J to verifying that expander assembly 10 is properly calibrated for use with the corresponding die set. As a result, tray assembly 370J, shown in FIG. 29, can be formed as simply comprising tray 372J having die set 92J mounted thereon, i.e., low calibration ring 374J and high calibration ring 376J and the complementary slots and/or projections for mounting them on tray 372J can be eliminated. The other tray assemblies 370 can be similarly formed.

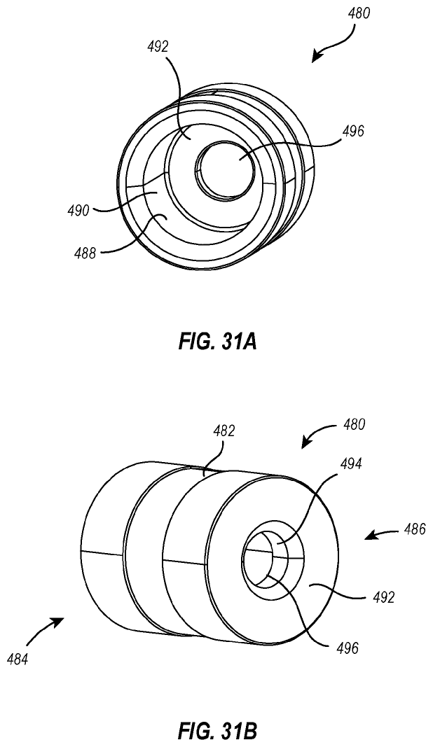

Depicted in FIGS. 31A and 31B is an exemplary calibration ring 480 that can be used to verifying that expander assembly 10 is properly calibrated for use with corresponding die sets. Calibration ring 480 comprises a tubular body 482 that extends between a first end 484 and an opposing second end 486. Tubular body 482 has a cylindrical interior surface 488 at first end 484 that encircles a first passage 490. An annular shoulder 492 radially inwardly projects from interior surface 488 at second end 486. Annular shoulder 492 has a cylindrical interior surface 494 that encircles a second passage 496 having a diameter that is smaller than the diameter of first passage 490. Although not required, in this embodiment, first passage 490 communicates with second passage 496. Calibration ring 480 in essence forms two separate calibration rings that are coupled together. Specifically, interior surface 488 at first end 484 forms an enlarged first calibration ring for use with larger diameter die sets 92 while interior surface 494 at second end 486 forms a smaller second calibration ring for use with smaller diameter die sets 92. In alternative embodiments, calibration ring 480 can be separated into two separate calibration rings, such as calibration rings 374J and 376J shown in FIG. 29. However, as discussed below, in this embodiment each die set 92 only needs one calibration ring for use in the verification process. Using calibration ring 480 in the verification process minimizes parts and thus simplifies the verification process.

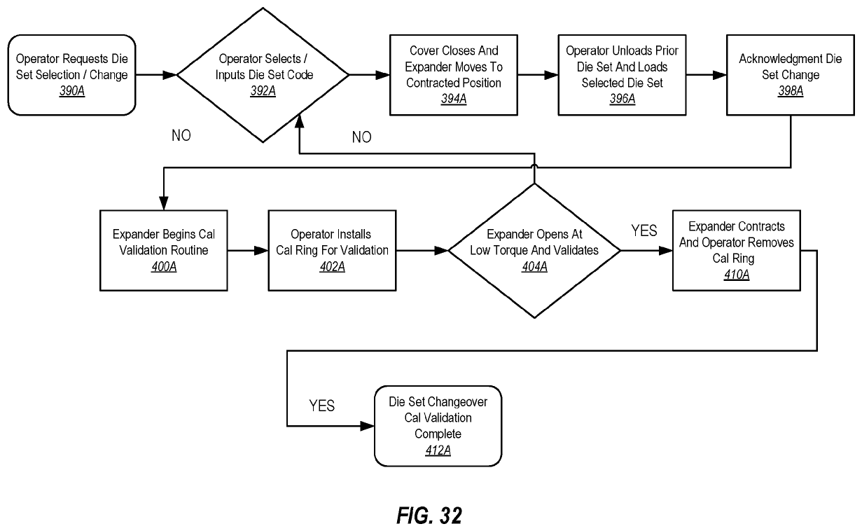

FIG. 32 is a flow chart providing an alternative example of how to validate the calibration of expander assembly 10, shown in FIG. 1, when using a select one of die set 92A-92J, shown in FIG. 29, and calibration ring 480, shown in FIGS. 31A and 31B. In step 390A, an operator activates expander assembly 10 and requests die set selection/change. All inputs by the operator can be through touch input on display screen 44 and/or through control switches 46. In step 392A, the operator is prompted for and either selects or inputs into expander assembly 10 a code corresponding to a select die set 92 chosen from die sets 91A-92J. The selected die set is chosen based on the desired size of compression collar 60 to be used. In the present example, die set 92J is the selected die set. The code can comprise an alpha and/or a numeric code with is typically labeled directly on each die set. See, for example, code 382J corresponding to “10” on die set 92J in FIG. 29. In step 394A, following the selection/input of the die set code, expander assembly 10 is programmed to automatically move cover 54 to the closed position (see FIG. 1) and, if not already done, move expander mechanism 90 to the contracted position, as shown in FIG. 6. In step 396A, cover 54 is then automatically moved to the open position (see FIG. 2) and the operator manually reaches down through access opening 30 and removes any die set currently mounted on expansion mechanism 90. The operator then grasps selected die set 92J, manually passes it down through access opening 30, and mounts it on expansion mechanism 90, as previously discussed with regard to FIG. 20 and shown in FIG. 6. As previously discussed, die sets 92 can be moved by using placement tool 450 (see FIG. 34) to sequentially move each of dies 96. In step 398A, one or more sensors within housing 12 of expander assembly 10 can be used to automatically detect that the selected die set 92J has been properly placed on expansion mechanism 90. For example, as shown in FIG. 5, optical sensors 386A and/or 386B, such as laser micrometers, can be used to detect the presence of the newly mounted die set 92J and confirm that it has been properly mounted. The placement of the selected die set 92J can be acknowledged on display screen 44.

In step 400A, expander assembly 10 now begins a calibration validation routine that is specific for selected die set 92J. In step 402A, the operator is prompted to and facilitates mounting of calibration ring 480 on die head 126J of die set 92J. More specifically, with cover 54 in the open position, the operator manually passes calibration ring 480 down access opening 30 and slides calibration ring 374 over die head 126J so that die head 126J is received within first passage 490 of calibration ring 480. Whether first passage 490 or second passage 496 of calibration ring 480 is used to receive the die head 126 is dependent on which die set 92 is being used. Specifically, as noted above, first passage 490 can be used with the larger die heads 126 while second passage 496 can be used with smaller die heads 126. Expander assembly 10 can dictate which passage 490, 496 to use based on input values of the die set 92 being used.

In step 404A, the operator can input that calibration ring 480 has been installed and/or sensors 386A and/or 386B can detect mounting thereof. Cover 54 then moves to the closed position and expansion mechanism 90 is automatically operated to outwardly expand die set 92J a predetermined distance. Specifically, expander assembly 10 using motor 154 automatically radially expands die set 92J a predetermined distance that is equal to the distance that die set 92J would expand when expanding a predefined compression collar 60 designed to operate with die set 92J. Expander assembly 10 then measures, directly or indirectly, the force being applied by die set 92J against the interior surface 488 of calibration ring 480. In one embodiment, the applied force can be measured by using a servo motor as motor 154. Expander assembly 10 then compares the measured force to a known predetermined force that die set 92J would actually be applying to the predefined compression collar 60 when expanding the predefined compression collar 60. If the compared forces are within a predefined tolerance, expander assembly 10 is properly calibrated for expanding compression collars using die set 92J. If not, expander assembly 10 is not properly calibrated for expanding compression collars using die set 92J. If the validation process fails, the process can return to step 392A to repeat the validation process or further recalibration steps can be taken.

If the validation process is successful, the process moves to step 410A where die set 92J is moved to the contracted position and calibration ring 480 is removed. In step 412A the die set change over calibration is complete. Once the validation process is completed and satisfied as outlined above, calibration for expander assembly 10 using die set 92J is also verified through extrapolation for expanding other predetermined compression collars 60 having a size for expanding on die set 92J. Such other predetermined compression collars 60 can be at least 1, 2, 3, 4, or more other compression collars. As a result, the validation process is not required for all compression collars being expanded by the same die set. If it is desired to expand a compression collar 60 that is designed to operate with a different sized die set, the above process steps 390A-412A are repeated for the new die set.

With expander assembly 10 having die set 92J mounted thereon and properly validated, the expansion of a compression collar is now discussed in association with the flow chart shown in FIG. 31. Specifically, in step 420, the operator inputs into expanding assembly 10 a code corresponding to the compression collar to be expanded. Only those codes corresponding to the compression collars approved for expanding by the mounted die set, i.e., die set 92J, will be accepted. The code for the desired compression collar can be manually input by the operator or can be scanned by using scanner 48 (FIG. 1) to scan a bar code or other scannable code located on the desired compression collar. The present example is assuming the mounting of compression collar 60A. However, it is understood that the mounted compression collar can be of any configuration disclosed herein that is sized to be expanded by die set 92J. If not already opened, cover 54 is moved to the open position and the operator manually passes the selected compression collar 60A down through access opening 30 and onto die set 92J. Specifically, compression collar 60A is typically advanced onto die head 126J until compression collar 60A sits firmly against the base of die head 126J (see FIG. 21). Compression collar 60A can be configured so that tabs 318A and 318B project above the top end face of die head 126J or they can be positioned against the side face of die head 126J. In step 422, once compression collar 60 is manually positioned on die head 126J, the operator inputs to start the expansion process. Next, in step 424, cover 54 is automatically moved to the closed position and, in subsequently step 426, sensors 386A and/or 386B validate that compression collar 60A is present and/or properly positioned on die set 92j/due head 126J. In addition, sensors 386A and/or 386B can also detect the outer diameter of the installed compression collar 60A to confirm if it matches the desired compression collar that was previously input. If any of the foregoing fail, in step 428 cover 54 is moved to the open position and the operator is prompted to correct the deficiency, after which the above process is repeated.

If the conditions of step 426 are satisfied, process moves to steps 428 wherein expansion mechanism 90 in association with die set 92J is used to radially expand the mounted compression collar 60A a predetermined radial distance based on the size of compression collar 60A. Compression collar 60A is expanded at a predetermined rate and is held in the expanded position by die set 92J for a predetermined period of time. Expanding a compression collar too fast can produce unwanted stress and/or unwanted plastic deformation on the compression collar. Furthermore, holding the compression collar in the expanded position for a period of time helps to delay the elastic rebound back toward the original unexpanded state, thereby providing the operator sufficient time to implement the desired use of the compression collar. In one exemplary embodiment, the rate of expansion of compression collars can be between 1 mm/s and 15 mm/s with between 2 mm/s and 10 mm/s or between 3 mm/s and 8 mm/s being more common. Other rates of expansion can also be used. The rate of expansion can vary depending on the size of the compression collar where smaller diameter compression collars are typically expanded at a slower rate than larger diameter compression collars. The period of time that compression collars are held in the expanded state is typically in a range between 0.2 seconds and 5 seconds with between 0.2 seconds and 3 seconds or between 0.2 seconds and 1 second being more common. Other values for the above can also be used depending on the facts.

If any of the above expanding conditions are determined by expander assembly 10 to not be satisfied, die set 92J is moved to the retracted position, cover 54 is moved to the open position, and expander assembly 10 prompts the operator in step 430 to remove the rejected compression collar 60 and place in scrap bin 80. Specifically, with reference to FIGS. 1 and 5, the operator manually removes rejected compression collar 60A from die set 92J and deposits it in collection opening 70. Rejected compression collar 60A travels down collection tube 72 and is deposited in scrap bin 80. Sensor 84 can detect the presence of rejected compression collar 60A which further ensures proper validation. The above process steps are then repeated with a new compression collar 60A.

If the conditions of step 428 are satisfied, sensors 386A and/or 386B can be used in step 430 to detect the outer diameter of the mounted compression collar 60A while being held in the expanded state. The detected outer diameter is compared by expander assembly 10 to a predetermined expected value for the outer diameter to confirm that compression collar 60A has been properly expanded. If the measured outer diameter is not within a predetermined tolerance of the expected value, prior discussed step 430 is applied and the rejected compression collar 60A is removed and transferred to the scrap bin 80 via collection opening 70. If the expansion conditions are satisfied, in step 432 expander assembly 10/expansion mechanism 90 moves die set 92J to the contracted position. In step 436, cover 54 is moved to the opened positioned and a removal timer is activated on display screen 44. In one embodiment, the removal timer is set for 10 seconds. However, other time limits such as at least 2, 4, 6, 8, 10, 12, 16, or 20 seconds or in a range between any two of the foregoing can be used depending on the current facts. The timer is part of the validation process and helps to ensure that the expanded compression collar is being promptly removed and used. Delay in removal and use can result in the compression collar resiliently constricting to such an extent that the compression collar cannot be properly applied to the tube and tube fitting. If compression collar 60A is not removed within the prescribed time, as determined by sensors 386A and/or 386B, the operator is again prompted to discard rejected compression collar 60A into scrap bin 80. The above-described step 430 is then repeated. In step 438, however, the operator properly removes expanded compression collar 60A within the described time period and is used to properly secure a tube to a tube fitting, as previously discussed. The process can then be repeated for further compression collars.

The use of expander assembly 10 for expanding compression collars and the above methods have a number of unique benefits. For example, expander assembly 10 can repeatedly, uniformly and accurately expand a plurality of different sizes of compression collars so as to ensure that they will all effect a desired seal. Expander assembly 10 can easily adapt to expanding a variety of different-sized compression collars. Expander assembly 10 can be implemented using repeatable validation steps to ensure that proper compression collars are being used and that they are being properly expanded. Expander assembly 10 is designed to minimize moving parts and thereby decrease complexity and minimize expense. In addition, expander assembly 10 can be designed to facilitate rotation through the use of bearings. The bearings enable the use of a low input force to facilitate rotation and can eliminate the need for external lubrication, which is conducive for operating expander assembly 10 within a clean room. Other benefits also exist.

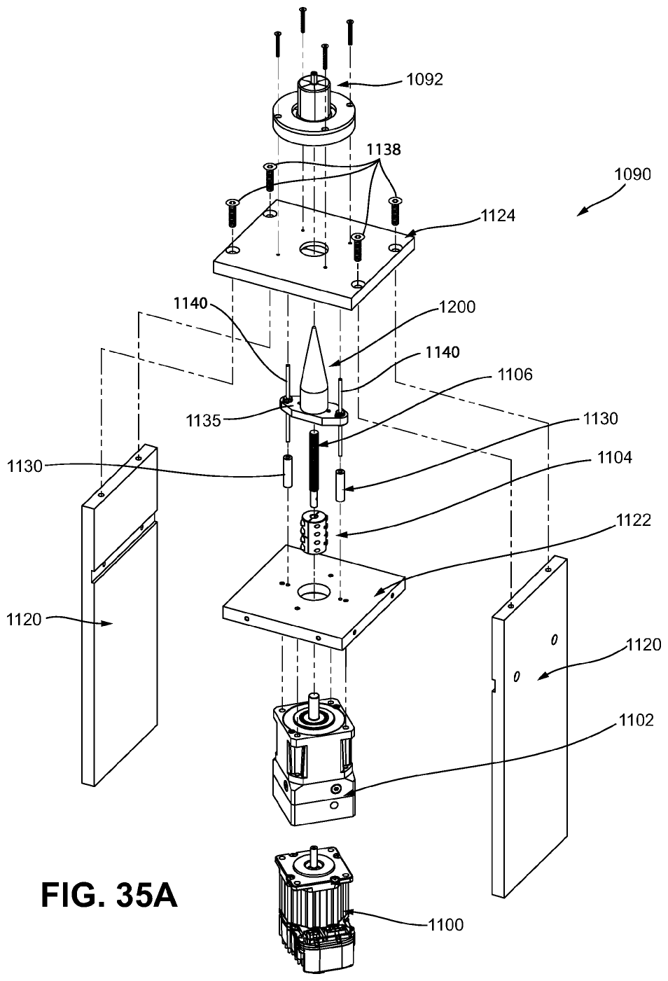

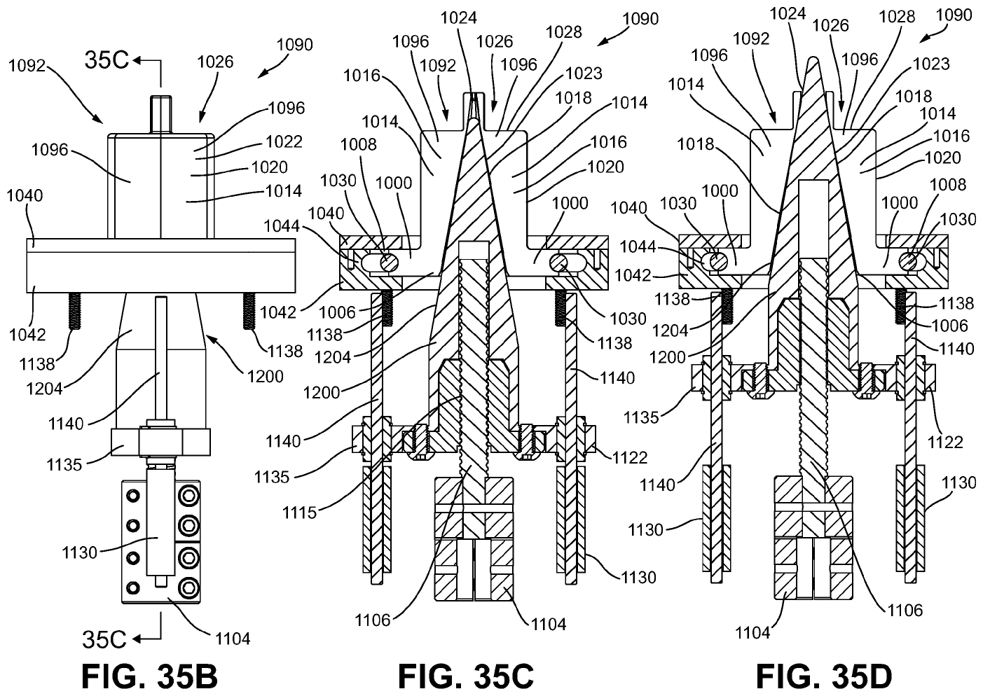

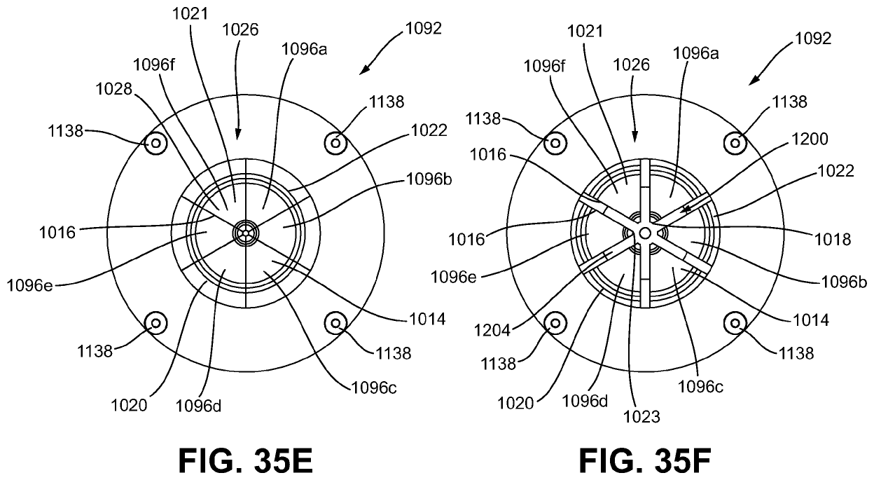

FIGS. 35A-35F illustrate an embodiment of an expansion mechanism 1090 that can be included in an embodiment of the expander assembly 10 (FIG. 1). For example, the expansion mechanism 1090 can be secured to the frame 36 and/or housing 12 illustrated in FIG. 5 (e.g., expansion mechanism can replace the embodiment of the expansion mechanism 90 shown in FIG. 5). However, the expansion mechanism 1090 can be secured and incorporated in any number of a variety of expander assemblies without departing from the scope of this disclosure.

As shown in FIG. 35A, the expansion mechanism 1090 can include an embodiment of a die set 1092 removeably mounted thereon. The combination of the expansion mechanism 1090 and die set 1092 can be used to selectively expand an embodiment of a compression collar 60A (FIG. 21). For example, compression collar 60A (FIG. 21) can be disposed on die set 1092 while die set 1092 is in a contracted position (as shown in FIGS. 35C and 35E). The expansion mechanism 1090 can be used to radially expand die set 1092 to an expanded position (as shown in FIGS. 35D and 35F) which in turn facilitates radial expansion of compression collar 60A (as shown in FIG. 22). Any one or more of the components and operation of the expansion mechanism 90 and die set 92 described above can be included in the expansion mechanism 1090 and die set 1092 described with respect to FIGS. 35A-35F and are not repeated here for sake of brevity.

For example, die set 1092 can include a plurality of dies 1096a-1096f (FIG. 35E) that are separate and discrete from each other and radially spaced about an axis. In one embodiment, each die 1096 can comprise an elongated base 1000 that extends between a first end 1006 and an opposing second end 1008, as depicted in FIGS. 35C and 35D. Upwardly expanding from first end 1006 of each base 1000 is a prong 1014. Each prong 1014 can be elongated and include a wedge-shaped transverse cross section that includes opposing side faces 1016 that converge toward an inside face 1018 and diverge toward an opposing outside face 1020 (also shown, for example, in FIG. 35B). The opposing side faces 1016 can extend between the inside face 1018 and the opposing outside face 1020. The outside face 1020 can be formed with a curvature. In one embodiment, opposing side faces 1016 are planar, although not required. Inside face 1018 can include a rounded face that can conform to and/or slidably engage an outer surface of a tapered expander, as will be described in greater detail below.

Outside face 1020 typically has the configuration of a smooth, continuous curve that extends or is disposed between side faces 1016, as shown in FIGS. 35E and 35F. In one exemplary embodiment, outside face 1020 forms an arc of a circle. Outside face 1020 is the surface that pushes against an interior surface of compression collar 60A (FIG. 21) during radial expansion of compression collar 60A, such as described above. Each of faces 1016, 1018, and 1020 extend upwardly from first end 1006 of base 1000 to a top face 1021 (FIGS. 35E and 35F), which can be planar.

When die set 1092 is in the contracted position, as shown in FIGS. 35C and 35E, side faces 1016 of each die 1096 are adjacently disposed to or are directly abutting against an opposing side face 1016 of an adjacent die 1096 so that prongs 1014 combine to form a die head 1026 having a cylindrical configuration and a circular transverse cross section. Die head 1026 has an encircling outside face 1022 formed from the combination of outside faces 1020 and terminates at a top end face 1028 (FIG. 35E) formed by the combination of top faces 1021. Outside face 1022 (FIG. 35E) also has a cylindrical configuration and a circular transverse cross section. Die head 1026, when in this contracted state (FIGS. 35C and 35E), is configured to receive compression collar 60A thereon (such as shown in FIG. 21).

Die head 1026 can also be formed with a circumferential inside face 1023, formed from the combination of inside faces 1018, that encircles or extends about a passage 1024, as shown in FIGS. 35C and 35D. As also shown in FIGS. 35C and 35D, each die 1096 can include an inside face 1018 that is tapered such that the inside face 1018 angles inward from the top end face 1028 towards the elongated base 1000. Additionally, each inside face 1018 can be rounded such that the plurality of tapered inside faces 1018 form a conical circumferential inside face 1023. The tapered and/or conical circumferential inside face 1023 can be flat or include other surface features that can slidably engage an expander to assist with expanding the die head 1026. As will be described in greater detail below, the tapered and/or conical circumferential inside face 1023 can be sized and shaped to conform to and/or slidably engage with a tapered expander (e.g., a conical shaped expander) that assists with expanding the die head 1026.

For example, as shown in FIG. 35A, the expansion mechanism 1090 can include a tapered expander 1200 that can be linearly translated between a first position (for example, as shown in FIG. 35C) and a second position (for example, as shown in FIG. 35D). The tapered expander 1200 can include an outer surface 1204 that increases in cross-sectional diameter between a first end and a second end of the tapered expander 1200. In the first position, the tapered expander 1200 can be positioned such that the outer surface 1204 of the tapered expander 1200 is not in contact with the circumferential inside face 1023 and/or is not applying a force against the circumferential inside face 1023 that results in movement (e.g., radial expansion) of the die head 1026. In the second position, the outer surface 1204 of the tapered expander 1200 can be in contact with the circumferential inside face 1023 and can apply a radial force against the circumferential inside face 1023 to thereby cause the die head 1026 to expand (FIGS. 35D and 35F), such as to expand the collar 60 (as shown in FIG. 22).

As the tapered expander 1200 linearly translates between the first position and the second position, the outer surface 1204 of the tapered expander 1200 can slidably engage and advance along the circumferential inside face 1023. For example, the tapered expander 1200 can have a conical shape and thus the outer surface 1204 can be conical. As the cross-sectional diameter of the tapered expander 1200 in contact with the inside face 1023 increases as the tapered expander 1200 translates into the second position, a radial force applied against the circumferential inside face 1023 can increase. For example, once the radial force exceeds an expansion force threshold, the die head 1026 can radially expand. In some embodiments, the expansion force threshold can be defined based on a spring or compressible material that assists in forming and biasing the die head 1026 in the compressed configuration. As shown in FIGS. 35C and 35D, a compressible material 1030 (e.g., O-ring, spring) can be positioned adjacent the second end 1008 of each die 1096 to thereby bias each die 1096 toward the center axis and bias the die head 1026 into the compressed configuration (FIGS. 35C and 35E). As such, when the tapered expander 1200 is linearly translating into the second position, as the cross-sectional diameter of the tapered expander 1200 in contact with the inside face 1023 increases and the radial force against the circumferential inside face 1023 increases, the die head 1026 can expand once the radial force becomes greater than a force required to compress the compressible material 1030.

As shown in FIGS. 35B-35D, the expansion mechanism 1090 can include an upper plate 1040 and a lower plate 1042 that forms a part of an embodiment of a compression collar expander assembly (e.g., compression collar expander assembly of FIG. 1). For example, one or more fastening elements 1138 (e.g., screws, etc.) can releasably secure the upper plate 1040 to the lower plate 1042, as shown in FIGS. 35A and 35B. In some embodiments, each base 1000 of each die 1096 can be at least partly captured between the upper plate 1040 and the lower plate 1042, as shown in FIGS. 35C and 35D. The base 1000 can be allowed to slidably translate between the upper plate 1040 and lower plate 1042, such as to allow the die head 1026 to form the expanded and compressed configurations. In some embodiments, the upper plate 1040 and/or the bottom plate 1042 can include a ledge that forms a sidewall 1044, as shown in FIGS. 35C and 35D. For example, one or more compressible material 1030 can be positioned between the sidewall 1044 and the second end 1008 of each die 1096, thereby allowing the compressible material 1030 to be compressed between each die 1096 and the sidewall 1044. In some embodiments, for example, the compressible material 1030 can include an O-ring that extends around the die head 1026, such as along an outer perimeter of the second end 1008 of each die 1096 or along an outer circumference of the base 1000 of each die 1096. As the tapered expander 1200 applies a radial force against the circumferential inside face 1023 that is greater than a compression force of the compressible material 1030 (e. g, a force required to compress the O-ring), the compressible material 1030 can be compressed between the die head 1026 and the sidewall 1044. In some embodiments, as the tapered expander 1200 moves into the first position and the radial force is decreased, the compressible material 1030 (e.g., O-ring) can be allowed to decompress thereby radially compressing and/or forcing the die head 1026 back to the compressed configuration (FIG. 35C).

As shown in FIG. 35A, the expansion mechanism 1090 can include mechanical and structural features that can assist with at least mounting the expansion mechanism 1090 (e.g., such as in system 10 of FIG. 1) and/or linearly translating the tapered expander 1200 to cause the tapered expander 1200 to expand the die head 1026. For example, as shown in FIG. 35A, the expansion mechanism 1090 can include and/or be coupled to a motor 1100 and a gearhead 1102 that assist with linearly translating the tapered expander 1200. As also shown in FIGS. 35A and 35B, the expansion mechanism 1090 can include and/or be coupled to a shaft coupling 1104 and a ball screw 1106 that assist with coupling the tapered expander 1200 to the gearhead 1102 and motor 1100. As such, activation of the motor 1100 and gearhead 1102 can cause the ball screw 1106 to rotate. The ball screw 1106 can be threadably engaged with a threaded inner channel 1115 (FIGS. 35C and 35D) of the tapered expander 1200, and the tapered expander 1200 can be rotationally fixed such that rotation of the ball screw 1106 in a first direction causes the tapered expander 1200 to linearly translate along a longitudinal axis towards and into the second position (for example, as shown in FIG. 35D). Rotation of the ball screw 1106 in a second direction can cause the tapered expander 1200 to linearly translate along the longitudinal axis towards and into the first position (for example, as shown in FIG. 35C).

As shown in FIG. 35A, the expansion mechanism 1090 can include one or more plates that can assist with mounting and positioning one or more parts of the expansion mechanism 1090. For example, as shown in FIG. 35A, the expansion mechanism 1090 can include one or more side plates 1120 that can secure to one or more sides of a motor mount plate 1122 (e.g., for securing either or both of the gearhead 1102 or the motor 1100) and a top plate 1124 (e.g., for securing the die set 1092). In some embodiments, one or more spacers 1130 and linear guides 1140 (FIGS. 35A and 35C) can extend between and/or through a part of the tapered expander 1200 (e.g., one or more flanges 1135 (see, for example, FIGS. 35A and 35C)) and the motor mount plate 1122. For example, the one or more linear guides 1140 can assist with preventing rotation of the tapered expander 1200 while allowing linear translation of the tapered expander 1200 along the one or more linear guides 1140. Other features and functions of the expansion mechanism 1090 illustrated in FIGS. 35A-35F can be appreciated from the figures and in light of the disclosure herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A compression collar expander assembly for expanding a tubular compression collar used in coupling a tube to a tube fitting, the compression collar expander assembly comprising:
a guide plate having a top surface and an opposing bottom surface with a plurality of lower guide channels extending therethrough;
a spindle plate disposed adjacent to the bottom surface of the guide plate, the spindle plate being rotatable relative to the guide plate and having a top surface with an expansion channel formed thereon;
a plurality of carriages movably disposed on the top surface of the guide plate, the plurality of carriages comprising at least three carriages that are radially spaced about an axis extending through the guide plate and are radially aligned with the axis, each of the plurality of carriages comprising:
a body; and
a guide projecting from the body, through a corresponding one of the lower guide channels formed on the guide plate, and into the expansion channel; and
a die set comprising a plurality of dies, each die comprising a base coupled to a corresponding one of the plurality of carriages and a prong upstanding from the base.

2. The compression collar expander assembly as recited in claim 1, wherein the expansion channel has a spiral configuration that encircles the axis.

3. The compression collar expander assembly as recited in claim 1, wherein each prong has a wedge-shaped configuration with opposing side faces that extend between an inside face and an opposing outside face, the outside face being formed with a curvature.

4. The compression collar expander assembly as recited in claim 3, wherein the curvature of the outside face is an arc of a circle.

5. The compression collar expander assembly as recited in claim 3, wherein the opposing side faces converge toward the inside face and diverge toward the outside face.

6. The compression collar expander assembly as recited in claim 1, wherein the plurality of dies comprises at least 3, 5, 7, 9, 11 or 13 separate dies.

7. The compression collar expander assembly as recited in claim 1, wherein each of the plurality of dies are separate from each other, are radially spaced about the axis, and are radially aligned with the axis.

8. The compression collar expander assembly as recited in claim 1, further comprising:
each of the plurality of carriages having a mounting hole formed thereon or a mounting post projecting therefrom; and
each of the plurality of dies having the other of the mounting hole or mounting post formed thereon, each mounting post being received with a corresponding mounting hole so as to couple each die to a corresponding carriage.

9. The compression collar expander assembly as recited in claim 1, wherein each carriage comprises a plurality of travel wheels rotatably mounted to the body and resting on the top surface of the guide plate.

10. The compression collar expander assembly as recited in claim 1, wherein the guide comprises:
a projection extending from the body and extending into the corresponding lower guide channel; and
a lower guide wheel rotatably mounted to the projection and at least partially disposed within the expansion channel.

11. The compression collar expander assembly as recited in claim 1, wherein the guide comprises:
an axle projecting from the body; and
a lower guide wheel rotatably mounted to the axle and at least partially disposed within the expansion channel.

12. The compression collar expander assembly as recited in claim 1, wherein the plurality of carriages comprises at least 5, 7, 9, 11 or 13 separate carriages.

13. The compression collar expander assembly as recited in claim 1, further comprising a cover plate secured to the guide plate so that the plurality of carriages are disposed between the guide plate and the cover plate.

14. The compression collar expander assembly as recited in claim 13, further comprising:
the cover plate having a top surface and an opposing bottom surface with a plurality of upper guide channels being formed on the bottom surface; and
each carriage having an upper guide wheel rotatably mounted to the body and disposed within a corresponding one of the plurality of upper guide channels.

15. The compression collar expander assembly as recited in claim 1, further comprising a motor coupled with the spindle plate, the motor being configured to rotate the spindle plate in opposite directions.

16.-50. (canceled)

51. A method for expanding a tubular compression collar used in coupling a tube to a tube fitting, the method comprising:
positioning the tubular compression collar on a die head of a die set so that the compression collar encircles the die head, the die head being comprised of a plurality of separate prongs radially spaced about an axis; and
radially displacing a plurality of carriages that are radially spaced about the axis so that the plurality of carriages move radially outward away from the axis, each of the plurality of carriages being coupled to a corresponding one of the plurality of prongs, the radial displacement of the plurality of carriages causing radial displacement of each of the plurality of prongs which in turn radially outwardly expands the tubular compression collar encircling the die head.

52. The method as recited in claim 51, wherein radially displacing the plurality of carriages comprises rotating a spindle plate about an axis of rotation, the spindle plate having a top surface with an expansion channel formed thereon, the rotation of the spindle plate causing the plurality of carriages to be displaced radially outward away from the axis.

53. The method as recited in claim 52, wherein rotating the spindle plate comprises activating a motor coupled with the spindle plate, the motor being configured to rotate the spindle plate.

54. The method as recited in claim 51, wherein each carriage comprises a body having a plurality of travel wheels rotatably mounted thereto.

55. The method as recited in claim 51, wherein the plurality of carriages comprises at least 5, 7, 9, 11 or 13 separate carriages.

56.-74. (canceled)
